SEQUENCE LISTING (45 sequences)

SEQ ID NO: 1 - DNA, 43 bp, Homo sapiens
  misc_feature: human sequence
cccccrgcatcccccctgcagcccccccagcatctcccctgca

SEQ ID NO: 2 - DNA, 20 bp, Artificial Sequence
  5-HTTLPR forward Taqman Primer
caactccctgtacccctcct

SEQ ID NO: 3 - DNA, 18 bp, Artificial Sequence
  5-HTTLPR Reverse Taqman Primer
tgcagggggatgctggaa

SEQ ID NO: 4 - DNA, 20 bp, Artificial Sequence
  Probe 1 Fam
tcctgcatcccccattatcc

SEQ ID NO: 5 - DNA, 17 bp, Artificial Sequence
  Probe 2 Vic
agcccccccagcatctc

SEQ ID NO: 6 - DNA, 20 bp, Artificial Sequence
  Rs25531 Forward Primer
caactccctgtacccctcct

SEQ ID NO: 7 - DNA, 17 bp, Artificial Sequence
  Rs25531 Reverse Primer
gagatgctgggggggct

SEQ ID NO: 8 - DNA, 14 bp, Artificial Sequence
  Rs25531 Fam
cctgcacccccagc

SEQ ID NO: 9 - DNA, 14 bp, Artificial Sequence
  Rs25531 Vic
ctgcacccccggca

SEQ ID NO: 10 - DNA, 437 bp, Homo sapiens
  misc_feature: human sequence
cgctctgaatgccagcacctaacccctaatgtccctactgcagccctcccagcatcccccctgcaacctcccagcaactccctgtacccctcctaggatcgctcctgcatcccccattatcccccccttcacccctcgcggcatcccccctgcacccccrgcatcccccctgcagcccccccagcatctcccctgcacccccagcatcccccctgcagcccttccagcatccccctgcacctctcccaggatctcccctgcaacccccattatcccccctgcacccctcgcagtatcccccctgcaccccccagcatccccccatgcacccccggcatcccccctgcacccctccagcattctccttgcaccctaccagtattcccccgcatcccggcctccaagcctcccgcccaccttgcggtccccgccctggc

SEQ ID NO: 11 - DNA, 20 bp, Artificial Sequence
  Repeat Sequence
ctcccctgcacccccagcat

SEQ ID NO: 12 - DNA, 21 bp, Artificial Sequence
  Repeat Sequence
cccccctgcaacctcccagca

SEQ ID NO: 13 - DNA, 23 bp, Artificial Sequence
  Repeat Sequence
actccctgtacccctcctaggat

SEQ ID NO: 14 - DNA, 22 bp, Artificial Sequence
  Repeat Sequence
cgctcctgcatcccccattatc

SEQ ID NO: 15 - DNA, 23 bp, Artificial Sequence
  Repeat Sequence
ccccccttcacccctcgcggcat

SEQ ID NO: 16 - DNA, 20 bp, Artificial Sequence
  Repeat Sequence
cccccctgcacccccrgcat

SEQ ID NO: 17 - DNA, 23 bp, Artificial Sequence
  Repeat Sequence
cccccctgcagcccccccagcat

SEQ ID NO: 18 - DNA, 20 bp, Artificial Sequence
  Repeat Sequence
ctcccctgcacccccagcat

SEQ ID NO: 19 - DNA, 22 bp, Artificial Sequence
  Repeat Sequence
cccccctgcagcccttccagca

SEQ ID NO: 20 - DNA, 23 bp, Artificial Sequence
  Repeat Sequence
tccccctgcacctctcccaggat

SEQ ID NO: 21 - DNA, 21 bp, Artificial Sequence
  Repeat Sequence
ctcccctgcaacccccattat

SEQ ID NO: 22 - DNA, 23 bp, Artificial Sequence
  Repeat Sequence
cccccctgcacccctcgcagtat

SEQ ID NO: 23 - DNA, 22 bp, Artificial Sequence
  Repeat Sequence
cccccctgcaccccccagcatc

SEQ ID NO: 24 - DNA, 20 bp, Artificial Sequence
  Repeat Sequence
cccccatgcacccccggcat

SEQ ID NO: 25 - DNA, 22 bp, Artificial Sequence
  Repeat Sequence
cccccctgcacccctccagcat

SEQ ID NO: 26 - DNA, 22 bp, Artificial Sequence
  Repeat Sequence
tctccttgcaccctaccagtat

SEQ ID NO: 27 - DNA, 531 bp, Homo sapiens
  misc_feature: human sequence
cgctctgaatgccagcacctaacccctaatgtccctactgcagccctcccagcatcccccctgcaacctcccagcaactccctgtacccctcctaggatcgctcctgcatcccccattatcccccccttcacccctcgcggcatcccccctgcacccccagcatcccccctgcagcccccccagcatctcccctgcacccccagcatcccccctgcagcccttccagcatccccctgcacctctcccaggatctcccctgcaacccccattatcccccctgcacccctcgcagtatcccccctgcaccccccagcatccccccatgcacccccggcatcccccctgcacccctccagcattctccttgcaccctaccagtattcccccgcatcccggcctccaagcctcccgcccaccttgcggtccccgccctggcgtctaggtggcaccagaatcccgcgcggactccacccgctgggagctgccctcgcttgcccgtggttgtccagctcagtccctctagacgctca

SEQ ID NO: 28 - DNA, 488 bp, Homo sapiens
  misc_feature: human sequence
cgctctgaatgccagcacctaacccctaatgtccctactgcagccctcccagcatcccccctgcaacctcccagcaactccctgtacccctcctaggatcgctcctgcatcccccattatcccccccttcacccctcgcggcatcccccctgcacccccagcatcccccctgcagcccttccagcatccccctgcacctctcccaggatctcccctgcaacccccattatcccccctgcacccctcgcagtatcccccctgcaccccccagcatccccccatgcacccccggcatcccccctgcacccctccagcattctccttgcaccctaccagtattcccccgcatcccggcctccaagcctcccgcccaccttgcggtccccgccctggcgtctaggtggcaccagaatcccgcgcggactccacccgctgggagctgccctcgcttgcccgtggttgtccagctcagtccctctagacgctca

SEQ ID NO: 29 - DNA, 20 bp, Artificial Sequence
  forward primer sequence
ctccctgtacccctcctagg

SEQ ID NO: 30 - DNA, 20 bp, Artificial Sequence
  forward primer sequence
ctccctgtacccctcctagg

SEQ ID NO: 31 - DNA, 17 bp, Artificial Sequence
  reverse primer sequence - Table 1
agaggtgcagggggatg

SEQ ID NO: 32 - DNA, 18 bp, Artificial Sequence
  reverse primer sequence - Table 1
ggtgcaggggagatgctg

SEQ ID NO: 33 - DNA, 18 bp, Artificial Sequence
  reverse primer sequence - Table 1
ggtgcaggggagatgctg

SEQ ID NO: 34 - DNA, 18 bp, Artificial Sequence
  reverse primer sequence - Table 1
ggtgcaggggagatgctg

SEQ ID NO: 35 - DNA, 20 bp, Artificial Sequence
  forward primer sequencer - Table 1
ctccctgtacccctcctagg

SEQ ID NO: 36 - DNA, 20 bp, Artificial Sequence
  forward primer sequencer - Table 1
ctccctgtacccctcctagg

SEQ ID NO: 37 - DNA, 20 bp, Artificial Sequence
  forward primer sequencer - Table 1
ctcccagcaactccctgtac

SEQ ID NO: 38 - DNA, 16 bp, Artificial Sequence
  reverse primer sequencer - Table 1
ctgggggtgcagggga

SEQ ID NO: 39 - DNA, 14 bp, Artificial Sequence
  5_HTTLR.a_V VIC probe sequence - Table 2
ccagcatctcccct

SEQ ID NO: 40 - DNA, 22 bp, Artificial Sequence
  5_HTTLPR.c_V VIC probe sequence - Table 2
tgcagcccccccagcatctccc

SEQ ID NO: 41 - DNA, 13 bp, Artificial Sequence
  rs25531_V VIC probe sequence - Table 2
tgcacccccggca

SEQ ID NO: 42 - DNA, 15 bp, Artificial Sequence
  5_HTTLR.a_F FAM Probe sequence - Table 2
cttcacccctcgcgg

SEQ ID NO: 43 - DNA, 28 bp, Artificial Sequence
  5_HTTLPR.c_F FAM Probe sequence - Table 2
tcccccccttcacccctcgcggcatccc

SEQ ID NO: 44 - DNA, 13 bp, Artificial Sequence
  rs25531_F FAM Probe sequence - Table 2
ctgcacccccagc

SEQ ID NO: 45 - DNA, 20 bp, Artificial Sequence
  forward primer sequence - Table 1
ctcccagcaactccctgtac

(19) United States
(12) Patent Application Publication
(10) Pub. No.: US 2016/0090625 A1
(43) Pub. Date: Mar. 31, 2016

(54) METHOD AND SYSTEM TO PREDICT SSRI RESPONSE

(71) Applicant: PATHWAY GENOMICS CORPORATION, San Diego, CA (US)

(72) Inventors: Cindy Wang, San Diego, CA (US); Tanya Moreno, San Diego, CA (US); K. David Becker, San Diego, CA (US)

(21) Appl. No.: 14/769,354

(22) PCT Filed: Mar. 13, 2014
(86) PCT No.: PCT/US14/26704

§ 371 Date: Aug. 20, 2015

Related U.S. Application Data
(63) Continuation-in-part of application No. 13/917,486, filed on Jun. 13, 2013.
(60) Provisional application No. 61/798,687, filed on Mar. 15, 2013.

(51) Int. Cl. C12Q 1/68

(52) U.S. Cl. CPC C12Q 1/6883; C12Q 2600/156; C12Q 2600/106; C12Q 2600/172

(57) ABSTRACT

The present inventions relates to methods and assays to predict the response of an individual to an SSRI treatment and to a method to improve medical treatment of a disorder, which is responsive to treatment with a selective serotonin reuptake inhibitor (SSRI).

### FIELD OF THE INVENTION

The invention relates to methods and assays to predict the response of an individual to an SSRI treatment and to a method to improve medical treatment of a disorder, which is responsive to treatment with a selective serotonin reuptake inhibitor (SSRI).

### REFERENCE TO SEQUENCE LISTING, TABLE, OR COMPUTER PROGRAM LISTING

The present application is being filed along with a Sequence Listing in electronic format. The Sequence Listing is provided as an 10 kb file entitled PATHG_009WO_SEQLISTING.TXT, created on Mar. 13, 2014, providing in electronic format subject matter which was present in the disclosure as originally filed. The information in the electronic format of the Sequence Listing is incorporated herein by reference in its entirety.

### RELATED APPLICATIONS

The present application claims priority to U.S. application Ser. No. 13/917,486, filed Jun. 13, 2013, the contents of which are hereby incorporated by reference in their entirety, and to U.S. Provisional Patent Application Ser. No. 61/798,687, “Method And System To Predict SSRI Response”, filed Mar. 15, 2013, the contents of which are hereby incorporated by reference in their entirety.

### BACKGROUND OF THE INVENTION

Selective serotonin re-uptake inhibitors or serotonin-specific reuptake inhibitor (SSRIs) are a class of compounds typically used as antidepressants in the treatment of depression, anxiety disorders, and some personality disorders. Major depression is the most common among those disorders treated with an SSRI.

Major depressive disorder (MDD) is a complex disorder associated with various monoaminergic disturbances, abnormalities of endocrine regulation, alterations in sleep physiology, neuroanatomic abnormalities, and neurophysiologic changes (Drevets, W C. 1998. Functional Neuroimaging Studies of Depression: The Anatomy of Melancholia. Annu Rev Med. 49:341-61; Thase, M. E. (1997), Psychotherapy of refractory depressions. Depress. Anxiety, 5: 190-201; Manji H K, Drevets W C, Charney D S (2001) The cellular neurobiology of depression, Nature Medicine 7(5), 541-7; Ordway, G. A., Stockmeier, C. A., Meltzer, H. Y., Overholser, J. C., Jaconetta, S. and Widdowson, P. S. (1995), Neuropeptide Y in Frontal Cortex Is Not Altered in Major Depression. Journal of Neurochemistry, 65: 1646-1650; Moretti A, Gorini A, Villa R F. 2003. Review Affective disorders, antidepressant drugs and brain metabolism. Mol Psychiatry. 8(9):773-85; Sheline Y I. Neuroimaging studies of mood disorder effects on the brain. Biol Psychiatry. 2003; 54:338-52; Buysse D J. Insomnia, depression and aging. Assessing sleep and mood interactions in older adults. Geriatrics. 2004; 59:47-51). Selective serotonin reuptake inhibitors (SSRIs) are frequently used to treat MDD; however, it is now clear that a large percentage of patients suffering from MDD may not benefit from SSRI treatment (Thase M E. 2003. Effectiveness of Antidepressants: Comparative Remission Rates. J Clin Psychiatry. 64[suppl 2]:3-7). Evidence from family studies indicates that response to specific antidepressant treatments can be affected by genetic differences (Pare C M, Mack J W. Differentiation of two genetically specific types of depression by the response to antidepressant drugs. J Med Genet. 1971; 8:306-9; O'Reilly R L, Bogue L, Singh S M. Pharmacogenetic response to antidepressants in a multicase family with affective disorder. Biol Psychiatry. 1994; 36:467-71; Serretti A, Franchini L, Gasperini M, et al. Mode of inheritance in mood disorders families according to fluvoxamine response. Acta Psychiatr Scand. 1998; 98:443-50). Pharmacogenetics holds the potential to genetically predict who will and will not benefit from SSRIs (Catalano M. The challenges of psychopharmacogenetics. Am J Hum Genet. 1999; 65:606-10.; Pickar D, Rubinow K. Pharmacogenomics of psychiatric disorders. Trends Pharmacol Sci. 2001; 22:75-83. [PubMed]; Serretti A, Lilli R, Smeraldi E. Pharmacogenetics in affective disorders. Eur J Pharmacol. 2002; 438:117-28.). Moreover, delineating the genes that are associated with poor response to an SSRI may be valuable in identifying alternative patient-specific treatments, such as novel treatments or currently known and available therapies.

Until recently, the focus of pharmacogenetics has been on functional gene variants that may contribute to variable antidepressant exposure (ie, pharmacokinetics). Extensive information is now available regarding genetic variation in many important metabolic enzymes (for reviews see Brosen K. The pharmacogenetics of the selective serotonin reuptake inhibitors. Clin Investig. 1993; 71:1002-9.; Cohen L J, De Vane C L. Clinical implications of antidepressant pharmacokinetics and pharmacogenetics. Ann Pharmacother. 1996; 30:1471-80.; Steimer W, Muller B, Leucht S, et al. Pharmacogenetics: a new diagnostic tool in the management of antidepressive drug therapy. Clin Chim Acta. 2001; 308:33-41.; Daly A K. Pharmacogenetics of the major polymorphic metabolizing enzymes. Fundam Clin Pharmacol. 2003; 17:27-41.; Evans W E, McLeod H L. Pharmacogenomics—drug disposition, drug targets, and side effects. N Engl J Med. 2003; 348:538-49.; Mancama D, Kerwin R W. Role of pharmacogenomics in individualising treatment with SSRIs. CNS Drugs. 2003; 17:143-51.).

Other well-known disorders that can be treated with SSRI's are dysthymia, premenstrual dysphoric disorder, panic disorder, obsessive compulsive disorder, social phobia, post-traumatic stress disorder, generalized anxiety disorder, obesity and alcoholism (Schatzberg J. Clin Psychiatry, 61, Suppl 11: 9-17, 2000; Masand and Gupta, Harvard Rev Psychiatry, 7: 69-84, 1999). Evidence is accumulating that such drugs have also beneficial effects in less common disorders, such as trichotillomania, paraphilia and related disorders and borderline personality disorder. Benefits are also obtained with use of an SSRI in smoking cessation and in the control of addictive behavior.

In many individuals, it is not uncommon that SSRI treatments fail to have clear therapeutic results or has to be discontinued due to poor tolerance of side effects. Approximately one-third of patients with major depressive disorder fail to respond to a correctly delivered antidepressant treatment and only 20-30% achieve remission (Ferrier I N 1999 Treatment of major depression: is improvement enough? J Clin Psychiatry 60(Suppl 6)10-14). Known side effects of SSRI's are headache, nausea, appetite inhibition, agitation, sleep disturbance, and disturbance of sexual functions, such as anorgasmia and loss of libido. In practice the overall therapeutic result of a regularly applied SSRI treatment is the resultant of the improvement of the disorder and the burden of negative side effects. In view of the existence of alternatives to SSRI's for the treatment of disorders, treatment results can be improved when patients are selected for tolerance and chance of success of an SSRI. Patients at risk for negative side effects can be treated with a treatment other than a treatment with an SSRI.

It is a known assumption that the genetic make-up of a person can contribute to the individually different responses of persons to a medicine (Roses, Nature 405:857-865, 2000). Examples of genetic factors, which determine drug tolerance, are drug allergies and severely reduced metabolism due to genetic absence of suitable enzymes. A case of a lethal lack of metabolism due to cytochrome P-450 2D6 genetic deficiency is reported by Sallee et at J Child & Adolesc. Psychopharmacol, 10: 27-34, 2000. The metabolic enzymes in the liver occur in polymorphic variants, causing some persons to metabolize certain drugs slowly and making them at risk for side effects due to excessively high plasma drug levels.

A polymorphism in the upstream regulatory site for the SERT gene (SLC6A4) has been widely studied. This SERT polymorphism (serotonin transporter linked polymorphic region; 5-HTTLPR) involves the presence or absence of a 43 base-pair segment in the promoter region of the gene, which produces a long (L) or short (S) allele; a difference that can influence transcriptional activity (Heils A, Mossner R, Lesch K P. The human serotonin transporter gene polymorphism—basic research and clinical implication. J Neural Transm. 1997; 104:1005-14.; Lesch K P. Serotonin transporter and psychiatric disorders: listening to the gene. Neuroscientist. 1998; 4:25-34.). 5-HTTLPR has been associated with susceptibility to depression (Caspi et al 2003), although there is considerable heterogeneity between studies (Lotrich F E, Pollock B G, Ferrell R E. Polymorphism of the serotonin transporter: implications for the use of selective serotonin reuptake inhibitors. Am J Pharmacogenomics. 2001; 1:153-64.; Lotrich F E, Pollock B G. Meta-analysis of serotonin transporter polymorphisms and affective disorder. Psychiatr Genet. 2004). It has emerged that the 5-HTTLPR polymorphism not only influences antidepressant response to SSRI but also tolerability (Kato M, Serretti A. 2010. Review and meta-analysis of antidepressant pharmacogenetic findings in major depressive disorder. Mol Psychiatry 15:473-500). However, because of the similar redundancy of these repeats, it is often difficult to separate the two polymorphisms.

The S allele has also been associated with diminished response to several SSRIs as compared with the L allele in multiple studies (Arias B, Gasto C, Catalan R, et al. Variation in the serotonin transporter gene and clinical response to citalopram in major depression. Am J Med Genet. 2000; 96:536.; Pollock B G, Ferrell R E, Mulsant B H, et al. Allelic variation in the serotonin transporter promoter affects onset of paroxetine treatment response in late-life depression. Neuropsychopharmacology. 2000; 23:587-90.; Zanardi R, Benedetti F, Di Bella D, et al. Efficacy of paroxetine in depression is influenced by a functional polymorphism within the promoter of the serotonin transporter gene. J Clin Psychopharmacol. 2000; 20:105-6.; Rausch J L, Johnson M E, Fei Y-J, et al. Initial conditions of serotonin transporter kinetics and genotype: influence on SSRI treatment trial outcome. Biol Psychiatry. 2002; 51:723-32.; Yu Y-Y, Tsai S-J, Chen T-J, et al. Association study of the serotonin transporter promoter polymorphism and symptomatology and antidepressant response in major depressive disorders. Mol Psychiatry. 2002; 7:1115-19.; Arias B, Catalan R, Gasto C, et al. 5-HTTLPR polymorphism of the serotonin transporter gene predicts non-remission in major depression patients treated with citalopram in a 12-weeks follow up study. J Clin Psychopharmacol. 2003; 23:563-7.), although there are two exceptions in Asian populations (Kim D K, Lim S-W, Lee S, et al. Serotonin transporter gene polymorphism and antidepressant response. Neuroreport. 2000; 11:215-19., Ito K, Yoshida K, Sato K, et al. A variable number of tandem repeats in the serotonin transporter gene does not affect the antidepressant response to fluvoxamine. Psychiatry Res. 2002; 111:235-9.). The S allele may also increase vulnerability to SSRI side effects (Mundo E, Walker M, Cate T, et al. The role of serotonin transporter protein gene in antidepressant-induced mania in bipolar disorder: preliminary findings. Arch Gen Psychiatry. 2001; 58:539-44.; Murphy G M, Kremer C, Rodrigues H, et al. The apolipoprotein E epsilon4 allele and antidepressant efficacy in cognitively intact elderly depressed patients. Biol Psychiatry. 2003a; 54:665-73.). While the general finding of worse outcome in SSRI-treated patients with the S allele has been well replicated, discrepant reporting in several of these studies makes it difficult to determine the effect size of this polymorphism. Among issues to be further clarified is the effect of 5-HTTLPR in different ethnic populations; linkage disequilibrium with other polymorphisms in different ethnic populations; the effect size in different age groups and at different doses of SSRIs; delineating which depressive symptoms and side effects are influenced; and determining how this polymorphism interacts with other polymorphisms. Moreover, the role of other SLC6A4 polymorphisms remains comparatively unexamined (Lesch 1998; Battersby S, Ogilvie A D, Blackwood D H R, et al. Presence of multiple functional polyadenylation signals and a single nucleotide polymorphism in the 3′untranslated region of the human serotonin transporter gene. J Neurochem. 1999; 72:1384-8.; Michaelovsky E, Frisch A, Rockah R, et al. A novel allele in the promoter region of the human serotonin transporter gene. Mol Psychiatry. 1999; 4:97-9.; M. Nakamura, S. Ueno, A. Sano & H. Tanabe (2000). “The human serotonin transporter gene linked polymorphism (5-HTTLPR) shows ten novel allelic variants”. Molecular Psychiatry 5 (1): 32-38.; Ito et al 2002).

Although researchers commonly report the polymorphism with two variations: a short (“S”) and a long (“L”), it can be subdivided further. One such study found 14 different alleles were found in different populations [M. Nakamura, S. Ueno, A. Sano & H. Tanabe (2000). “The human serotonin transporter gene linked polymorphism (5-HTTLPR) shows ten novel allelic variants”. Molecular Psychiatry 5 (1): 32-38] In connection with the region are two single nucleotide polymorphisms (SNP) which contribute to this subdivision: rs25531 and rs25532. [L. Murphy & Klaus-Peter Lesch (February 2008). “Targeting the murine serotonin transporter: insights into human neurobiology”. Nature Reviews Neuroscience 9 (2): 85-86].

With the results from one study the polymorphism was thought to be related to treatment response so that long-allele patients respond better to antidepressants [L. Kathryn Durham, Suzin M. Webb, Patrice M. Milos, Cathryn M. Clary, Albert B. Seymour (August 2004). “The serotonin transporter polymorphism, 5HTTLPR, is associated with a faster response time to sertraline in an elderly population with major depressive disorder”. Psychopharmacology 174 (4): 525-529] Another antidepressant treatment response study did, however, rather point to the rs25531 SNP, [Jeffrey B. Kraft, Susan L. Slager, Patrick J. McGrath & Steven P. Hamilton (September 2005). “Sequence analysis of the serotonin transporter and associations with antidepressant response”. Biological psychiatry 58 (5): 374-381] and a large study by the group of investigators found a “lack of association between response to an SSRI and variation at the SLC6A4 locus”. [Jeffrey B. Kraft, Eric J. Peters, Susan L. Slager, Greg D. Jenkins, Megan S. Reinalda, Patrick J. McGrath & Steven P. Hamilton (March 2007). “Analysis of association between the serotonin transporter and antidepressant response in a large clinical sample”. Biological Psychiatry 61 (6): 734-742].

### SUMMARY OF THE INVENTION

The present invention is related to methods and systems to detect more accurately and rapidly the presence of a 43 bp insertion/deletion polymorphism in the 5′ regulatory region of the SLC6A4 gene in order to predict an individual's response to SSRI therapies. Particularly, the present invention is directed to the use of a unique identifier probe that uniquely identifies the two forms of polymorphisms found in the gene.

In one aspect, the method also requires isolating a sample containing the genetic material to be tested, and thereafter using genotyping techniques to differentiate the long and short form alleles of the SLC6A4 gene. In another aspect, the method also requires isolating a sample biological material containing proteomic information, epigenetic information, biochemical information, metagenomic information and thereafter using genotyping or other techniques to differentiate the long and short form alleles of the SLC6A4 gene.

These methods to identify gene expression levels are not limited by the technique that is used to identify the expression level of the gene of interest. Methods for measuring gene expression are well known in the art and include, but are not limited to, immunological assays, nuclease protection assays, northern blots, in situ hybridization, Polymerase Chain Reaction (PCR) such as reverse transcriptase Polymerase Chain Reaction (RT-PCR) or Real-Time Polymerase Chain Reaction, expressed sequence tag (EST) sequencing, cDNA microarray hybridization or gene chip analysis, subtractive cloning, Serial Analysis of Gene Expression (SAGE), Massively Parallel Signature Sequencing (MPSS), and Sequencing-By-Synthesis (SBS).

After a patient has been identified as likely to be responsive to the therapy based on the identity of one or more of the genetic markers identified herein, the method may further comprise administering or delivering an effective amount of a SSRI treatment or an alternative treatment, to the patient, based on the outcome of the determination. Methods of administration of pharmaceuticals and biologicals are known in the art and are incorporated herein by reference.

It is conceivable that one of skill in the art will be able to analyze and identify genetic markers in situ at some point in the future. Accordingly, the inventions of this application are not to be limited to requiring isolation of the genetic material prior to analysis.

These methods also are not limited by the technique that is used to identify the polymorphism of interest. Suitable methods include but are not limited to the use of hybridization probes, antibodies, primers for PCR analysis, and gene chips, slides and software for high throughput analysis. Additional genetic markers can be assayed and used as negative controls.

This invention also provides a panel, kit, gene chip or software for patient sampling and performance of the methods of this invention. The kits contain gene chips, slides, software, probes or primers that can be used to amplify and/or for determining the molecular structure or expression level of the genetic markers identified above. Instructions for using the materials to carry out the methods are further provided.

This invention also provides for a panel of genetic markers selected from, but not limited to the genetic polymorphisms identified herein or in combination with each other. The panel comprises probes or primers that can be used to amplify and/or for determining the molecular structure of the polymorphisms identified above. The probes or primers can be used for all RT-PCR methods as well as by a solid phase support such as, but not limited to a gene chip or microarray. The probes or primers can be detectably labeled. This aspect of the invention is a means to identify the genotype of a patient sample for the genes of interest identified above.

The disclosure herein may be further understood by consideration of the following enumerated embodiments.

1. A method for determining an individual's response to selective serotonin reuptake inhibitor (SSRI) treatment, comprising genotyping the individual at an SLC6A4 locus for at least one 5-HTTLPR L allele.

2. The method according to embodiment 1, wherein said genotyping comprises analyzing a sample from the individual.

3. The method according to embodiment 2, wherein said samples is selected from blood, including serum, lymphocytes, lymphoblastoid cells, fibroblasts, platelets, mononuclear cells or other blood cells, from saliva, liver, kidney, pancreas or heart, urine or from any other tissue, fluid, cell or cell line derived from the human body.

4. The method according to embodiment 3, wherein said sample is saliva.

5. The method according to any one of embodiments 1-4, wherein the subject suffers from depression.

6. The method according to any one of embodiments 1-5, wherein the method comprises assaying for at least one copy of a 43 base-pair segment in the promoter region of the SLC6A4 gene having the sequence of SEQ ID NO:1.

7. The method according to embodiment 6, wherein the method comprises detecting the 43 base-pair segment with a probe having the sequence of SEQ ID NO:5.

8. The method of any one of embodiments 1-7, further comprising detecting an SLC6A4 locus with a probe comprising a sequence of SEQ ID NO:4.

9. The method according to any one of embodiments 1-8, comprising:

genotyping a nucleic acid sample from an individual to detect the presence or absence of at least one L allele of the SLC6A4 gene,

genotyping a nucleic acid sample from an individual to detect the SLC6A4 gene, and

comparing the level of the L allele to the level of the SLC6A4 gene.

10. The method according to embodiment 9, further comprising using a first probe unique to the L allele, and a second probe to detect the presence of the SLC6A4 gene, wherein the second probe is not unique to the L allele. In some embodiments the first probe and second probe are selected from a Table herein, such as Table 1.

11. The method according to embodiment 10, wherein the first probe comprises the nucleic acid sequence of SEQ ID NO: 5 and the second probe can be any probe to the SLC6A4 gene, more preferably a probe to the 5-HTTLPR in the SLC6A4 gene.

12. The method according to embodiment 10, wherein the second probe is to any portion of the 5-HTTLPR outside the 43 by insertion (SEQ ID NO:1).

13. The method of embodiment 12, wherein the second probe is a sequence of SEQ ID NO:4.

14. The method according to any one of embodiments 1-13, further comprising detecting a single nucleotide polymorphism (SNP) in the serotonin-transporter-gene-linked polymorphic region (5-HTTLPR).

15. The method according to embodiment 14, wherein the polymorphism is selected from rs25531 and rs25532.

16. The method according to any one of embodiments 1-15, wherein an individual having an equal amount of sample detected with a probe to said L allele as compared to an amount of sample detected with a probe to said SCL6A4 is categorized as being homozygous for said L allele, and wherein an individual having an unequal amount of sample detected with a probe to said L allele as compared to an amount of sample detected with a probe to said SCL6A4 is categorized as not being homozygous for said L allele.

17. The method of embodiment 16, wherein said individual identified as having homozygous L alleles is characterized as an individual more likely to be responsive to SSRI treatments.

18. The method according to embodiment 16, wherein the individual is identified as having average or above average response to SSRI treatments, reduced vulnerability to side effects, or increased tolerance to SSRI treatment in comparison to similarly situated individuals whose genotypes are not homozygous for the L allele.

19. The method according to embodiment 16, wherein a person identified as not having homozygous L alleles is characterized as an individual less likely to be responsive to SSRI treatments.

20. The method according to embodiment 19, wherein the individual is identified as having reduced response to SSRI treatments, increased vulnerability to side effects, or decreased tolerance to SSRI treatment in comparison to similarly situated individuals who are homozygous for the L allele.

21. The method according to embodiment 16, further comprising treating the individual with non-SSRI therapies if the patient is not homozygous for the L allele.

22. The method according to embodiment 16, further comprising treating the patient with an SSRI if the patient is homozygous for the L allele.

23. The method of embodiment 22, wherein the SSRI is selected from the list consisting of fluoxetine, fluvoxamine, citalopram, cericlamine, dapoxetine, escitalopram, femoxetine, indalpine, paroxetine, sertraline, paroxetine, ifoxetine, cyanodothiepin, zimelidine, and litoxetine.

24. The method according to embodiment 16, further comprising reducing the effective dosage of SSRI if the patient is homozygous for the L allele.

25. A genetic test for assessing an individual's response to SSRI therapies, comprising a first probe specific to a sequence of SEQ ID NO:1 and a second probe specific to a sequence of SEQ ID NO:4.

26. The genetic test of embodiment 25, wherein said first probe comprises a sequence of SEQ ID NO:5.

27. The genetic test of embodiment 25, wherein the individual's saliva is tested as the sample.

28. A method of selecting a mental health treatment for an individual comprising the steps of assaying for SLC6A4 L allele status, and selecting an SSRI pharmaceutical if said allele status is homozygous for an SLC6A4 L allele, and selecting a non-SSRI pharmaceutical if said allele status is not homozygous for an SLC6A4 L allele.

29. The method of embodiment 28, wherein said SSRI pharmaceutical is selected from the list consisting of fluoxetine, fluvoxamine, citalopram, cericlamine, dapoxetine, escitalopram, femoxetine, indalpine, paroxetine, sertraline, paroxetine, ifoxetine, cyanodothiepin, zimelidine, and litoxetine.

30. The method of any of embodiments 28-29, wherein said assaying for SLC6A4 status comprises obtaining a sample from said individual, and contacting said sample with a first probe that detects an L allele having a sequence of SEQ ID NO:1, and contacting said second sample with a second probe that detects said SLC6A4 locus outside of SEQ ID NO:1.

31. The method of embodiment 30, wherein said first probe comprises a sequence of SEQ ID NO:5.

32. The method of any one of embodiments 30-31, wherein said second probe comprises a sequence of SEQ ID NO:4.

33. The method of any of embodiments 28-32, further comprising detecting a single nucleotide polymorphism (SNP) in the serotonin-transporter-gene-linked polymorphic region (5-HTTLPR).

34. The method according to embodiment 33, wherein the polymorphism is selected from rs25531.

35. The method of embodiment 34, further comprising selecting a non-SSRI pharmaceutical if said allele status at rs25531 is the nucleic acid base “G” even if said sample is homozygous for the L allele.

36. The method of any of embodiments 28-29, wherein said assaying for SLC6A4 status comprises obtaining a sample from said individual, and contacting said sample with a first polymerase chain reaction primer pair that detects an L allele having a sequence of SEQ ID NO:1, and contacting said second sample with a second polymerase chain reaction primer pair that detects said SLC6A4 locus outside of SEQ ID NO:1.

37. The method of embodiment 36, wherein said first primer pair amplifies a sequence of SEQ ID NO:5.

38. The method of any one of embodiments 36-37, wherein said second primer pair amplifies a sequence of SEQ ID NO:4.

39. The method of any of embodiments 36-38, further comprising detecting a single nucleotide polymorphism (SNP) in the serotonin-transporter-gene-linked polymorphic region (5-HTTLPR).

40. The method according to embodiment 39, wherein the polymorphism is selected from rs25531.

41. The method of embodiment 34, further comprising selecting a non-SSRI pharmaceutical if said allele status at rs25531 is the nucleic acid base “G” even if said sample is homozygous for the L allele.

42. A method of predicting an individual's response to a selective serotonin reuptake inhibitor treatment individual comprising the steps of assaying for SLC6A4 L allele status, and selecting an SSRI pharmaceutical if said allele status is homozygous for an SLC6A4 L allele, and selecting a non-SSRI pharmaceutical if said allele status is not homozygous for an SLC6A4 L allele.

43. The method of embodiment 42, wherein said SSRI pharmaceutical is selected from the list consisting of fluoxetine, fluvoxamine, citalopram, cericlamine, dapoxetine, escitalopram, femoxetine, indalpine, paroxetine, sertraline, paroxetine, ifoxetine, cyanodothiepin, zimelidine, and litoxetine.

44. The method of any of embodiments 42-43, wherein said assaying for SLC6A4 status comprises obtaining a sample from said individual, and contacting said sample with a first probe that detects an L allele having a sequence of SEQ ID NO:1, and contacting said second sample with a second probe that detects said SLC6A4 locus outside of SEQ ID NO:1.

45. The method of embodiment 44, wherein said first probe comprises a sequence of SEQ ID NO:5.

46. The method of any one of embodiments 30-31, wherein said second probe comprises a sequence of SEQ ID NO:4.

47. The method of any of embodiments 42-46, further comprising detecting a single nucleotide polymorphism (SNP) in the serotonin-transporter-gene-linked polymorphic region (5-HTTLPR).

48. The method according to embodiment 47, wherein the polymorphism is selected from rs25531.

49. The method of embodiment 48, further comprising selecting a non-SSRI pharmaceutical if said allele status at rs25531 is the nucleic acid base “G” even if said sample is homozygous for the L allele.

50. The method of any of embodiments 42-43, wherein said assaying for SLC6A4 status comprises obtaining a sample from said individual, and contacting said sample with a first polymerase chain reaction primer pair that detects an L allele having a sequence of SEQ ID NO:1, and contacting said second sample with a second polymerase chain reaction primer pair that detects said SLC6A4 locus outside of SEQ ID NO:1.

51. The method of embodiment 50, wherein said first primer pair amplifies a sequence of SEQ ID NO:5.

52. The method of any one of embodiments 50-51, wherein said second primer pair amplifies a sequence of SEQ ID NO:4.

53. The method of any of embodiments 50-52, further comprising detecting a single nucleotide polymorphism (SNP) in the serotonin-transporter-gene-linked polymorphic region (5-HTTLPR).

54. The method according to embodiment 53, wherein the polymorphism is selected from rs25531.

55. The method of embodiment 54, further comprising selecting a non-SSRI pharmaceutical if said allele status at rs25531 is the nucleic acid base “G” even if said sample is homozygous for the L allele.

56. The method of any of embodiments 1-55, wherein said embodiment comprises the use of an oligonucleotide disclosed herein.

### BRIEF DESCRIPTION OF THE DRAWINGS

FIG. 1 shows complex sequence of the 5-HTTLPR region and location of the target of interest.

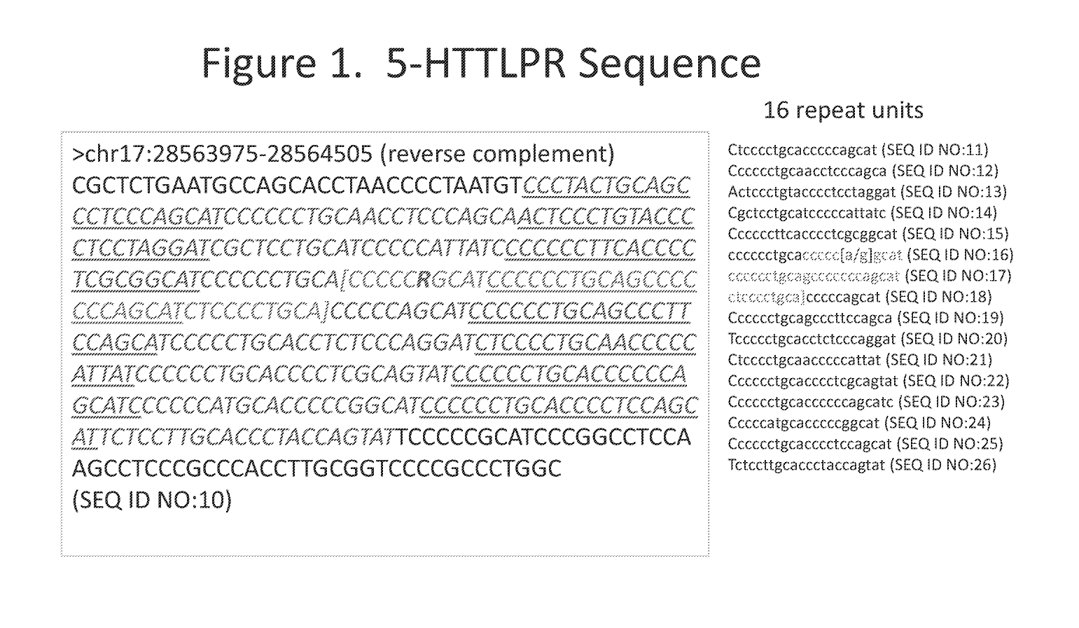

FIG. 2 shows the positions and sequences of the primers and probes for 5-HTTLPR assay use to detect the presence of a 43 by insertion/deletion polymorphism in the 5′ regulatory region and the two forms of polymorphisms (rs25531 (A/G SNP)) found in the SLC6A4 gene which indicated the specificity of the primers and probes as well as the uniqueness of the assay.

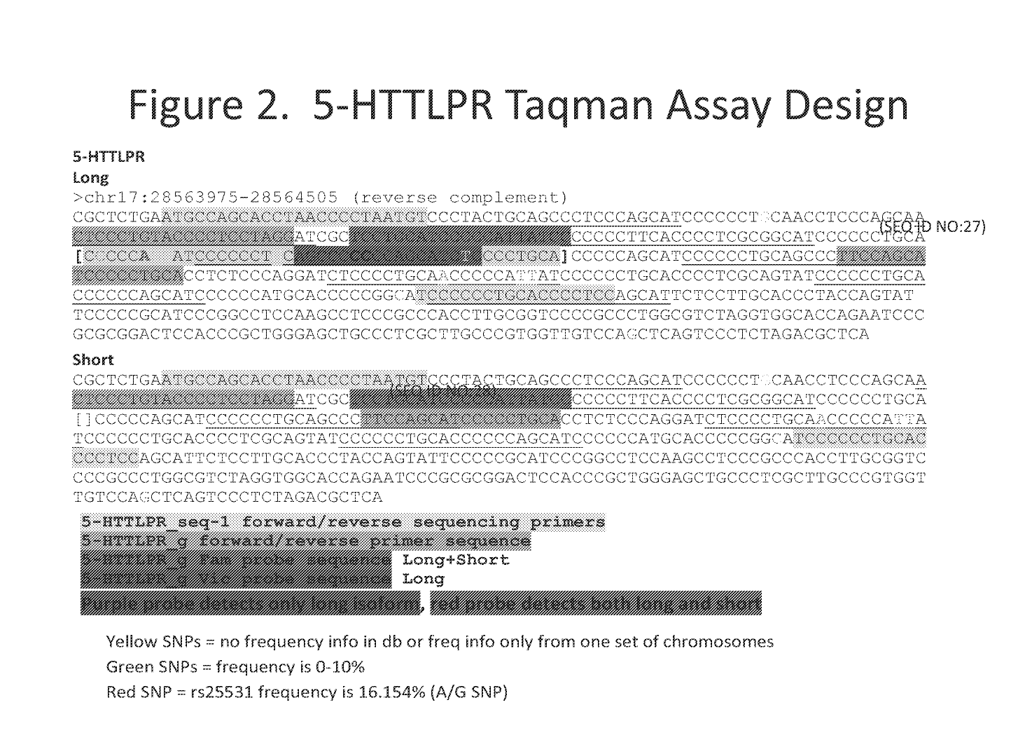

FIG. 3 shows the discordant between 5-HTTLPR PCR based genotyping assay (Fluidigm) and nucleic acid sequencing analysis due to the limitation of PCR method. PCR amplification favors the short allele (14 repeats), therefore generating predominately 14 repeat PCR products as targets for sequencing analysis. Highlighted heterozygous (L/S) (16 repeat:14 repeat) PCR amplified samples are scored as the rare homo (14 repeat:14 repeat) when sequenced

FIG. 4 illustrates novel TaqMan primers used in rs25531 assays were designed specific to target 16 or 14 repeats and the combination of the previous sequencing forward primer and 5-HTTLPR reverse Taqman primers could produce unique and evenly distributed PCR products (used for sequencing confirmation).

FIG. 5 shows PCR results comparison between Taqman primer pairs of assay rs25531_C and rs25531_J. rs25531_J assay primers could distinguish the long from the short allele of 5-HTTLPR but not rs25531_C assay primers. It also defined the rules for genotype calling algorisms throughout the pipeline.

### DETAILED DESCRIPTION OF THE PREFERRED EMBODIMENTS

Serotonin Transporter (5HTT, Locus Symbol SLC6A4 (solute carrier family 6, member 4)), which maps to 17q11.1-17q12 (Ramamoorthy S, Giovanetti E, Qian Y, et al. Phosphorylation and regulation of antidepressant-sensitive serotonin transporters. J Biol Chem. 1998; 273:2458-66.), contains a 43 by insertion/deletion (ins/del, 5HTTLPR) polymorphism in the 5′ regulatory region of the gene (Heils A, Teufel A, Petri S, Stober G, Riederer P, Bengel D, Lesch K P (June 1996). “Allelic variation of human serotonin transporter gene expression”. Journal of Neurochemistry 66 (6): 2621-2624) (SEQ. ID. No. 1: ccccc[a/g]gcatcccccctgcagcccccccagcatctcccctgca]. The ins/del in the promoter appears to be associated with variations in transcriptional activity: the long variant (L) has approximately three times the basal activity of the short promoter (S) with the deletion (Lesch et al., 1996), although this is not a universal finding (Willeit et al., 2001, Kaiser et al., 2002). The S variant has been reported to be dominant over the L variant (Heils et al., 1996), although at least one report suggests that the L may be dominant over the S (Williams, R. B., Marchuk, D. A., Gadde, K. M., Barefoot, J. C., Grichnik, K., Helms, M. J., Kuhn, C. M., Lewis, J. G., Schanberg, S. M., Stafford-Smith, M., Suarez, E. C., Clary, G. L., Svenson, I. K. and Siegler, I. C. (2003). Serotonin-related gene polymorphisms and central nervous system serotonin function. Neuropsychopharmacology 28: 533-541). Several investigators have reported that the 5-HTTLPR polymorphism affects serotonergic functions in vivo. Individuals with the L/L genotype were found to have significantly higher maximal uptake of serotonin into platelets compared to those with L/S or S/S genotypes (Nobile, M., Begni, B., Giorda, R., Frigerio, A., Marino, C., Molteni, M., Ferrarese, C., & Battaglia, M. J. (1999). Effects of serotonin transporter promoter genotype on platelet serotonin transporter functionality in depressed children and adolescents. Journal of the American Academy of Child and Adolescent Psychiatry 38: 1396-1402., Greenberg, B. D., Tolliver, T. J., Huang, S. J. Li, Q., Bengel, D., & Murphy D. L. (1999).).

The promotor region of the SLC6A4 gene contains a polymorphism with “short” and “long” repeats in the region: 5-HTT-linked polymorphic region (5-HTTLPR or SERTPR). [Heils A, Teufel A, Petri S, Stober G, Riederer P, Bengel D, Lesch K P (June 1996). “Allelic variation of human serotonin transporter gene expression”. Journal of Neurochemistry 66 (6): 2621-2624.] The short variation has 14 repeats of a sequence while the long variation has 16 repeats. [M. Nakamura, S. Ueno, A. Sano & H. Tanabe (2000). “The human serotonin transporter gene linked polymorphism (5-HTTLPR) shows ten novel allelic variants”. Molecular Psychiatry 5 (1): 32-38; although recent studies have identified longer repeats 17, and 18 repeats characterized with higher transcription levels similar to 16 repeats, and a third allele with 11 repeats that is functionally comparable with the short allele (S) Ehli E A, Hu Y, Lengyel-Nelson T, Hudziak J J, Davies G E. 2011. Identification and functional characterization of three novel alleles for the serotonin transporter-linked polymorphic region. Mol Psychiatry (Presented at The American Society of Human Genetics 60th Annual Meeting, Washington D.C., Nov. 2-6, 2010)]. Although the polymorphism is described as a 43 bp insertion/deletion, the present invention does not depend on the exact size of the insertion/deletion polymorphism, but rather the existence or absence of a nucleic acid fragment that is highly redundant with other repeats in this region. Accordingly, the polymorphism can be longer or shorter, e.g., 44, 45, 46, 47, 48, 49, 42, 41, 40, 39, 38, 37, or longer or shorter, for example, 2× longer, 86, etc.

The short variation leads to less transcription for SLC6A4, and it has been found that it can partly account for anxiety-related personality traits. [Lesch K P, Bengel D, Heils A, Sabol S Z, Greenberg B D, Petri S, Benjamin J, Müller C R, Hamer D H, Murphy D L (November 1996). “Association of Anxiety-Related Traits with a Polymorphism in the Serotonin Transporter Gene Regulatory Region”. Science 274 (5292): 1527-31] This polymorphism has been extensively investigated in several hundred scientific studies. [Wendland J R, Martin B J, Kruse M R, Lesch K P, Murphy D L (2006). “Simultaneous genotyping of four functional loci of human SLC6A4, with a reappraisal of 5-HTTLPR and rs25531”. Molecular Psychiatry 274 (3): 1-3.] The 5-HTTLPR polymorphism may be subdivided further: One study published in 2000 found 14 allelic variants (14-A, 14-B, 14-C, 14-D, 15, 16-A, 16-B, 16-C, 16-D, 16-E, 16-F, 19, 20 and 22) in a group of around 200 Japanese and Caucasian people [M. Nakamura, S. Ueno, A. Sano & H. Tanabe (2000). “The human serotonin transporter gene linked polymorphism (5-HTTLPR) shows ten novel allelic variants”. Molecular Psychiatry 5 (1): 32-38]. The difference between 16-A and 16-D is the rs25531 SNP. It is also the difference between 14-A and 14-D. [J. R. Wendland, B. J. Martin, M. R. Kruse, Klaus-Peter Lesch, D. L. Murphy (2006). “Simultaneous genotyping of four functional loci of human SLC6A4, with a reappraisal of 5-HTTLPR and rs255531” (PDF). Molecular Psychiatry 274 (3): 1-3]. Other studies have shown correlation with other psychiatric diseases (including mood disorders, autism, panic disorder, schizophrenia, Alzheimer's disease, obsessive compulsive disorder, personality traits and depressive symptomatology in mood disorders) with diverse findings (see, e.g., Furlong R A et al. Am J Med Genet 1998. 81: 58-63; Collier D A et al. Mol Psychiatry 1996. 1: 453-460, Lesch K P et al. Science 1996. 274: 1527-1531, Kunugi H et al. Lancet 1996. 347: 1340, Ebstein R P et al. Mol Psychiatry 1997. 2: 224-226, Klauck S M et al. Hum Mol Genet 1997. 6: 2233-2238, Deckert J et al. Psychiatr Genet 1997. 7: 45-47, Billet E A et al. Mol Psychiatry 1997. 2: 403-406, Hoeche M R et al. Am J Med Genet 1998. 81: 1-3, Esterling L E et al. Am J Med Genet 1998. 81: 37-40, Katsugari S et al. Biol Psychiatry 1999. 45: 368-370, Bengel D et al. Mol Psychiatry 1999. 4: 463-466].

In addition to altering the expression of SERT protein and concentrations of extracellular serotonin in the brain, the 5-HTTLPR variation is associated with changes in brain structure. One study found less grey matter in perigenual anterior cingulate cortex and amygdala for short allele carriers of the 5-HTTLPR polymorphism compared to subjects with the long/long genotype. [Pezawas L, Meyer-Lindenberg A, Drabant E M, Verchinski B A, Munoz K E, Kolachana B S, Egan M F, Mattay V S, Hariri A R, Weinberger D R (June 2005). “5-HTTLPR polymorphism impacts human cingulate-amygdala interactions: a genetic susceptibility mechanism for depression”. Nature Neuroscience 8 (6): 828-34]

The structure and function of the 5HTTLPR is more complex. Because of the variations in the 5-HTTLPR region, 5HTT gene is multiallelic. A SNP in the promoter region of the serotonin transporter gene (rs25531; A/G) transformed the 5HTTLPR into a triallelic locus (Hu X Z, Lipsky R H, Zhu G, Akhtar L A, Taubman J, Greenberg B D, et al. 2006. Serotonin transporter promoter gain-of-function genotypes are linked to obsessive-compulsive disorder. Am J. Hum Genet 78:815-826.). The LG and the S alleles showed comparable levels of serotonin transporter expression, both of which were inferior to the LA, which was associated with a lesser side effect burden (Hu X Z, Rush A J, Charney D, Wilson A F, Sorant A J, Papanicolaou G J, et al. 2007. Association between a functional serotonin transporter promoter polymorphism and citalopram treatment in adult outpatients with major depression. Arch Gen Psychiatry 64:783-792).

Described herein are methods and systems for rapidly and accurately genotyping a person for the presence of particular markers in the 5-HTTLPR region of the 5HTT (SLC6A4) gene. Because 5HTTLPR is a multi-allelic locus (Hu X Z, Lipsky R H, Zhu G, Akhtar L A, Taubman J, Greenberg B D, et al. 2006. Serotonin transporter promoter gain-of-function genotypes are linked to obsessive-compulsive disorder. Am J. Hum Genet 78:815-826.), it is often difficult to rapidly or accurately assess the genotype of a person without cumbersome, multi-step assays or full sequencing of the entire region. The present invention describes genotyping a person for the presence of the particular polymorphisms in a 43 bp insertion/deletion DNA sequence in the 5HTT receptor gene means screening patients to determine the type and number of 5HTT receptor alleles present in the patient. Such screening may be carried out by various methods including nucleic acid sequencing of DNA. For example, the screening may be accomplished by restriction isotyping methods, which include the general steps of polymerase chain reaction amplification, restriction digestion, and gel electrophoresis. Screening may also be carried out by other types of nucleic acid sequencing, e.g., by hybridization or oligotyping, or by direct sequencing of DNA nucleotides. The advantage of this invention is less turnaround time and gets both 5-HTTLPR and rs25531 genotype results at the same time when and improves accuracy when compared to other methods.

Before the compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

Throughout this disclosure, various publications, patents and published patent specifications are referenced by an identifying citation. The disclosures of these publications, patents and published patent specifications are hereby incorporated by reference in their entirety into the present disclosure to more fully describe the state of the art to which this invention pertains.

### DEFINITIONS

The terms “genetic variation” or “genetic variant”, as they are used in the present description include mutations, polymorphisms and allelic variants. A variation or genetic variant is found amongst individuals within the population and amongst populations within the species.

The term “polymorphism” refers to a variation in the sequence of nucleotides of nucleic acid where every possible sequence is present in a proportion of equal to or greater than 1% of a population. A portion of a gene of which there are at least two different forms, i.e., two different nucleotide sequences, is referred to as a “polymorphic region of a gene”. A polymorphic region can be a single nucleotide, the identity of which differs in different alleles; in a particular case, when the said variation occurs in just one nucleotide (A, C, T or G) it is called a single nucleotide polymorphism (SNP).

A “polymorphic gene” refers to a gene having at least one polymorphic region.

The term “genetic mutation” refers to a variation in the sequence of nucleotides in a nucleic acid where every possible sequence is present in less than 1% of a population.

The terms “allelic variant” or “allele” are used without distinction in the present description and refer to a polymorphism that appears in the same locus in the same population.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “genotype” refers to the specific allelic composition of an entire cell or a certain gene, whereas the term “phenotype’ refers to the detectable outward manifestations of a specific genotype.

As used herein, the term “gene” or “recombinant gene” refers to a nucleic acid molecule comprising an open reading frame and including at least one exon and (optionally) an intron sequence. The term “intron” refers to a DNA sequence present in a given gene which is spliced out during mRNA maturation.

As used herein, the term “haplotype” refers to a group of closely linked alleles that are inherited together.

The expression “amplification” or “amplify” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

“Biological sample” or “sample” refers to the biological sample that contains nucleic acid taken from a fluid or tissue, secretion, cell or cell line derived from the human body. For example, samples may be taken from blood, including serum, lymphocytes, lymphoblastoid cells, fibroblasts, platelets, mononuclear cells or other blood cells, from saliva, liver, kidney, pancreas or heart, urine or from any other tissue, fluid, cell or cell line derived from the human body. For example, a suitable sample may be a sample of cells from the buccal cavity.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous”sequence shares less than 40% identity, though preferably less than 25% identity, with one of the sequences of the present invention.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence that has a certain degree of homology with or with the complement thereof. In one aspect, homologs of nucleic acids are capable of hybridizing to the nucleic acid or complement thereof.

The term “interact” as used herein is meant to include detectable interactions between molecules, such as can be detected using, for example, a hybridization assay. The term interact is also meant to include “binding” interactions between molecules. Interactions may be, for example, protein-protein, protein-nucleic acid, protein-small molecule or small molecule-nucleic acid in nature.

The term “isolated” as used herein with respect to nucleic acids, such as DNA or RNA, refers to molecules separated from other DNAs or RNAs, respectively, which are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments that are not naturally occurring as fragments and would not be found in the natural state. The term “isolated” is also used herein to refer to polypeptides that are isolated from other cellular proteins and is meant to encompass both purified and recombinant polypeptides.

The term “mismatches” refers to hybridized nucleic acid duplexes that are not 100% homologous. The lack of total homology may be due to deletions, insertions, inversions, substitutions or frameshift mutations.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or RNA, the terms “adenosine”, “cytidine”, “guanosine”, and “thymidine” are used. It is understood that if the nucleic acid is RNA, a nucleotide having a uracil base is uridine.

The terms “oligonucleotide” or “polynucleotide”, or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

As used herein, the term “label” intends a directly or indirectly detectable compound or composition that is conjugated directly or indirectly to the composition to be detected, e.g., polynucleotide so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6 ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. HANDBOOK OF FLUORESCENT PROBES AND RESEARCH CHEMICALS (6 ed.). (1996).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succi nimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

When a genetic marker or polymorphism “is used as a basis” for selecting a patient for a treatment described herein, the genetic marker or polymorphism is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits. As would be well understood by one in the art, measurement of the genetic marker or polymorphism in a clinical setting is a clear indication that this parameter was used as a basis for initiating, continuing, adjusting and/or ceasing administration of the treatments described herein.

The term “treating” as used herein is intended to encompass curing as well as ameliorating at least one symptom of the condition or disease.

A “response” implies any kind of improvement or positive response either clinical or non-clinical such as, but not limited to, measurable evidence of diminishing disease or disease progression, complete response, partial response, stable disease, increase or elongation of progression free survival, increase or elongation of overall survival, or reduction in toxicity or side effect vulnerability.

The term “likely to respond” shall mean that the patient is more likely than not to exhibit at least one of the described treatment parameters, identified above, as compared to similarly situated patients.

As used herein, the terms “increased”, “higher”, “greater” or similar terms in association with the ability of individuals with a certain genotype to respond to a SSRI shall mean having average or above average response to SSRI treatments, reduced vulnerability to side effects, or increased tolerance to SSRI treatment in comparison to similarly situated individuals with different genotype(s) regarding the L/S polymorphism. Alternatively, the terms “decreased”, “lower”, “reduced” or similar terms in association with the ability of individuals with a certain genotype to respond to an SSRI shall mean having less or reduced response to SSRI treatments, increased vulnerability to side effects, or reduced tolerance to SSRI treatment in comparison to similarly situated individuals with different genotype(s) regarding the L/S polymorphism. In this regard, the L/L homozygous genotype is indicative of a predisposition to a positive response to a SSRI drug.

In one embodiment, the present invention relates to a method of genotyping genetic variations in an individual, which is sufficiently sensitive, specific and reproducible as to allow its use in a clinical setting. The inventors have developed unique methodology with specifically designed primers and probes for use in the method.

Thus in one aspect, the invention comprises an in vitro method for genotyping genetic variations in an individual. The in vitro, extracorporeal method is for simultaneous sensitive, specific and reproducible genotyping of multiple human genetic variations present in one or more genes of a subject. The method of the invention allows identification of nucleotide changes, such as, insertions, duplications and deletions and the determination of the genotype of a subject for a given genetic variation.

A given gene may comprise one or more genetic variations. Thus the present methods may be used for genotyping of one or more genetic variations in one or more genes.

Thus a genetic variation may comprise a deletion, substitution or insertion of one or more nucleotides. In one aspect the genetic variations to be genotyped according to the present methods comprise SNPs.

Typically the individual is a human.

Typically, for a given genetic variation there are three possible genotypes:

LL the individual is homozygous for genetic variation L (e.g homozygous for the L allele)
SS the individual is homozygous for genetic variation S (e.g. homozygous for the S allele)
LS the individual is heterozygous for genetic variations L and S (e.g. one L allele and one S allele)

By permitting clinical genotyping of one or more of the above genetic variations, the present method has use in for example, diagnosing susceptibility to or resistance to SSRI treatment or adverse reactions to SSRI treatment, e.g., pharmaceuticals.

At least one genetic variation is analyzed in the present methods. Thus the present methods may be used for genotyping an individual with respect to the L or S allele, as described herein.

One aspect of the present invention provides a method and an assay to determine an individuals likely response to SSRIs, said method comprising detecting at least one marker within 5HTTLPR in a sample derived from a subject, wherein the presence of at least one marker is indicative of an individuals likely response to SSRIs. Preferably, the marker that is identified is the long or short allele of the SLC6A4 gene.

In one embodiment, the present invention comprises predicting an individuals response to SSRI treatment, comprising:

a) Genotyping a nucleic acid sample from an individual to detect the presence or absence of at least one L allele of the SLC6A4 gene.

Preferably, the method comprises genotyping the individual for the presence of homozygous L alleles in the 5-HTTLPR in the SLC6A4 serotonin transporter gene.

In a more preferred embodiment, the present invention further comprises predicting an individual's response to SSRI treatment by screening the individual using genotyping methods, wherein a probe unique to the L allele is used to identify at least one L allele. More preferably, the probe comprises the nucleic acid sequence of SEQ ID NO: 5.

In a more preferred embodiment, the present invention comprises genotyping the individual for the presence of homozygous L alleles or S alleles, or heterozygous alleles of the L allele and the S allele. One such method for determining whether the individual carries homozygous or heterozygous alleles is to detect the presence of multiple L alleles, for example, determining the level of L alleles in comparison to the total number of SLC6A4 genes, or determining the level of L alleles in comparison to a person that is homozygous for L alleles or homozygous for S alleles. In a preferred embodiment of the present invention, there are provided methods and systems for determining the presence of multiple L alleles, comprising detecting the level of L alleles in comparison to the level of the SLC6A4 gene, more preferably, in comparison to the level of the 5-HTTLPR in the SLC6A4 serotonin transporter gene.

Accordingly, in one embodiment, the present invention comprises:

genotyping a nucleic acid sample from an individual to detect the presence or absence of at least one L allele of the SLC6A4 gene,

genotyping a nucleic acid sample from an individual to detect the SLC6A4 gene, in parallel or in sequence with detecting the at least one L allele, and

comparing the level of the L allele to the level of the SLC6A4 gene.

Wherein equal levels of the L allele and the SLC6A4 gene identify the presence of homozygous L alleles, and wherein the presence of the L allele but at a level lower than the level of the SLC6A4 gene identifies the presence of heterozygous L allele and S allele, and the detection of SLC6A4 but not the L allele identifies the presence of homozygous S alleles.

A preferred method for comparing the levels of the L allele comprises using a first probe unique to the L allele, and a second probe to detect the presence of the SLC6A4 gene, wherein the second probe is not unique to the L allele. More preferably, the first probe comprises the nucleic acid sequence of SEQ ID NO: 5. The second probe can be any probe to the SLC6A4 gene, more preferably a probe to the 5-HTTLPR in the SLC6A4 gene. In one example, the second probe is the nucleic acid sequence of SEQ ID NO:4, although it is contemplated that any probe to any portion of the 5-HTTLPR outside the 43 by insertion (SEQ ID NO: 1) can be used.

The invention further provides methods for detecting the single nucleotide polymorphism in the gene of interest. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

Mutations associated with the gene may result in changes in serotonin transporter function, and experiments with mice have identified more the 50 different phenotypic changes as a result of genetic variation. These phenotypic changes may, e.g., be increased anxiety and gut dysfunction. Some of the human genetic variations associated with the gene are:

Length variation in the serotonin-transporter-gene-linked polymorphic region (5-HTTLPR)

rs25531—a single nucleotide polymorphism (SNP) in the 5-HTTLPR
rs25532—another SNP in the 5-HTTLPR
STin2—a variable number of tandem repeats (VNTR) in the functional intron 2
G56A on the second exon
I425V on the ninth exon
Length variation in 5-HTTLPR
Main article: 5-HTTLPR

In a preferred embodiment, the present invention further comprises detecting the rs25531 single nucleotide polymorphisms found in the long form of 5-HTTPLR, and more preferably, the single marker is the single nucleotide substitution of an A for a G at the rs25531 SNP, designated as LA and LG, respectively. The more common LA allele is associated with the reported higher basal activity, whereas the less common LG allele is associated with having transcriptional activity no greater than the S allele.

### Diagnostic Methods

The invention further features diagnostic medicines, which are based, at least in part, on determination of the identity of the polymorphic region or expression level (or both in combination) of the genetic markers above.

For example, information obtained using the diagnostic assays described herein is useful for determining if a subject will respond to SSRI treatment for a given indication. Based on the prognostic information, a doctor can recommend a therapeutic protocol, useful for prescribing different treatment protocols for a given individual.

In addition, knowledge of the identity of a particular allele in an individual (the gene profile) allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. Expression patterns of individual patients can then be compared to the expression profile of the disease to determine the appropriate drug and dose to administer to the patient.

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

Genotyping of an individual can be initiated before or after the individual begins to receive SSRI treatment. Preferably, upon genotyping, the treatment of the individual can be adapted depending on the presence or absence of the L allele. The genotyping serves to decide on timely adapting the further treatment when the person is found to fall into the genotype category homozygous for the L allele of the SLC6A4 gene.

Side effects of an SSRI treatment are those related to SSRI treatment based on a positive correlation between frequency or intensity of occurrence and SSRI drug treatment. Such information is usually collected in the course of studies on efficacy of a drug treatment and many methods are available to obtain such data. Resulting information is widely distributed among the medical profession and patients receiving treatment. Specifically identified SSRI treatment related side effects are headache, dizziness, agitation, trouble concentrating, gastrointestinal disturbances, nausea, vomiting, diarrhea, appetite inhibition, sleep disturbance, somnolence, insomnia and disturbance of sexual functions, such as anorgasmia and loss of libido. Premature drug-related discontinuation of an SSRI treatment and drug-related adverse events are also included here within the definition of side effects of an SSRI treatment.

A treatment result is defined here from the point of view of the treating doctor, who judges the efficacy of a treatment as a group result. Within the group, individual patients can recover completely and some may even worsen, due to statistical variations in the course of the disease and the patient population. Some patients may discontinue treatment due to side effects, in which case no improvement in their condition due to SSRI treatment can occur. An improved treatment result is an overall improvement assessed over the whole group. Improvement can be solely due to an overall reduction in frequency or intensity of side effects. It is also possible that doses can be increased or the dosing regime can be stepped up faster thanks to less troublesome side effects in the group and consequently an earlier onset of recovery or better remission of the disease.

A disorder, which is responsive to treatment with an SSRI, is defined to be a disorder, which is, according to recommendations in professional literature and drug formularies, known to respond with at least partial remission of the symptoms to a treatment with an SSRI. In most countries such recommendations are subject to governmental regulations, allowing and restricting the mention of medical indications in package inserts. Other sources are drug formularies of health management organizations. Before approval by governmental agencies certain recommendations can also be recognized by publications of confirmed treatment results in peer reviewed medical journals. Such collective body of information defines what is understood here to be a disorder that is responsive to treatment with an SSRI. Being responsive to SSRI treatment does not exclude that the disorder in an individual patient can resist treatment with an SSRI, as long as a substantial portion of persons having the disorder respond with improvement to the SSRI treatment.

The main indication for SSRIs is clinical depression. SSRIs are also frequently prescribed for major depressive disorder, anxiety disorders such as social anxiety, dysphoric disorder, panic disorders, obsessive-compulsive disorder (OCD), dysthymia, premenstrual, eating disorders such as obesity, bulimia nervosa, chronic pain, alcoholism, trichotillomania, paraphilia and related disorders, borderline personality disorder, smoking cessation, drug abuse and occasionally, for posttraumatic stress disorder (PTSD) or depersonalization disorder.

The technical field defines an SSRI as a drug that inhibits the reuptake of serotonin in nerve terminals in the brain more effectively than the reuptake of noradrenaline and dopamine. Such drugs are treated as a group with common properties due to this mechanism of action and this selectivity. Known drugs specifically named as SSRI are fluoxetine, fluvoxamine, citalopram, cericlamine, dapoxetine, escitalopram, femoxetine, indalpine, paroxetine, sertraline, paroxetine, ifoxetine, cyanodothiepin, zimelidine, and litoxetine, of which fluoxetine, fluvoxamine, citalopram, sertraline and paroxetine are the ones with which most experience is obtained and which are preferred indicators for defining the SSRI responsive disorders for which the method according to this invention can be applied (Hermann, Canadian J Clin Pharmacol 7: 91-95, 2000; Modell et al., Clin Pharm & Ther 61: 476-487, 1997; Lucid et al., Neurosci biobehav. Rev 18: 85-95, 1994).

In an alternate method, the present invention comprises predicting an individual's response to alternate therapies, for example, repetitive transcranial magnetic stimulation to drug-resistant depression with long/long homozygotes benefitting more than short-allele carriers (Luisella Bocchio-Chiavetto, Carlo Miniussi, Roberta Zanardini, Anna Gazzoli, Stefano Bignotti, Claudia Specchia & Massimo Gennarelli (May 2008). “5-HTTLPR and BDNF Va166Met polymorphisms and response to rTMS treatment in drug resistant depression”. Neuroscience Letters 437 (2): 130-134). The selection of a therapy which is recognized to be an alternative for a treatment with an SSRI is defined by reference to the general knowledge in this medical field. It is standard practice to diagnose a disorder in a person and select a therapy that is indicated for the disorder. An example of a reference manual for diagnostic methods is the Diagnostic and Statistical Manual of Mental Disorders 4th edition (DSM-IV) published by the American Psychiatric Association, Washington, D.C. (1994). Supplementary to this the SSRI responsive disorders can be identified objectively on the basis of recommendations in government approved labels, by health management organizations and in confirmed reports of positive treatment results in peer reviewed medical literature, it is similarly known to the skilled person that alternative non-SSRI treatments are available. Examples of non-SSRI anti-depressant treatments are, for example, treatments with drugs, which act by blocking the reuptake of norepinephrine, or by blocking α2 adrenergic or serotonin receptors. Specific alternative antidepressants include venlafaxine, mirtazapine, duloxetine, bupropion, trazodone, buspirone, nefazodone, amitriptyline, nortriptyline, doxepine and imipramine. Alternative treatments for prescribing an SSRI can also be non-drug treatments, such as behavioral therapies or electroconvulsive shock treatment. Benzodiazepine-like anxiolytic compounds can be used for anxiety disorders. A therapy diminishing the risk for side effects of an SSRI treatment can also be a treatment with a. below average dose of the SSRI or providing an additional treatment to prevent side effects.

Various embodiments of the invention provide for methods for identifying a genetic variation (e.g, allelic patterns, polymorphism patterns such as SNPs, or haplotype patterns etc.), comprising collecting biological samples from one or more subjects and exposing the samples to detection assays under conditions such that the presence or absence of at least one genetic variation is revealed. To begin, polynucleotide samples derived from (e.g., obtained from) an individual may be employed. Any biological sample that comprises a polynucleotide from the individual is suitable for use in the methods of the invention. The biological sample may be processed so as to isolate the polynucleotide. Alternatively, whole cells or other biological samples may be used without isolation of the polynucleotides contained therein.

Detection of a genetic variation in a polynucleotide sample derived from an individual can be accomplished by any means known in the art, including, but not limited to, amplification of a sequence with specific primers; determination of the nucleotide sequence of the polynucleotide sample; hybridization analysis; single strand conformational polymorphism analysis; denaturing gradient gel electrophoresis; mismatch cleavage detection; and the like. Detection of a genetic variation can also be accomplished by detecting an alteration in the level of a mRNA transcript of the gene; aberrant modification of the corresponding gene, e.g., an aberrant methylation pattern; the presence of a non-wild-type splicing pattern of the corresponding mRNA; an alteration in the level of the corresponding polypeptide; determining the electrophoretic mobility of the allele or fragments thereof (e.g., fragments generated by endonuclease digestion), and/or an alteration in corresponding polypeptide activity.

In some embodiments, a subject can be genotyped for an allele, more preferably a polymorphism by collecting and assaying a biological sample of the patient to determine the nucleotide sequence of the gene at that polymorphism, the amino acid sequence encoded by the gene at that polymorphism, or the concentration of the expressed product, e.g., by using one or more genotyping reagents, such as but not limited to nucleic acid reagents, including primers, etc., which may or may not be labeled, amplification enzymes, buffers, etc. In certain embodiments, the target polymorphism will be detected at the protein level, e.g., by assaying for a polymorphic protein. In yet other embodiments, the target polymorphism will be detected at the nucleic acid level, e.g., by assaying for the presence of nucleic acid polymorphism, e.g., a single nucleotide polymorphism (SNP) that cause expression of the polymorphic protein. Any convenient protocol for assaying a sample for the above one or more target polymorphisms may be employed in the subject methods.

In general, nucleic acid is extracted from the biological sample using conventional techniques. The nucleic acid to be extracted from the biological sample may be DNA, or RNA, typically total RNA. Typically RNA is extracted if the genetic variation to be studied is situated in the coding sequence of a gene. Where RNA is extracted from the biological sample, the methods further comprise a step of obtaining cDNA from the RNA. This may be carried out using conventional methods, such as reverse transcription using suitable primers. Subsequent procedures are then carried out on the extracted DNA or the cDNA obtained from extracted RNA. The term DNA, as used herein, may include both DNA and cDNA.

In general the genetic variations to be tested are known and characterized, e.g. in terms of sequence. Therefore nucleic acid regions comprising the genetic variations may be obtained using methods known in the art.

In one aspect, DNA regions which contain the genetic variations to be identified (target DNA regions) are subjected to an amplification reaction in order to obtain amplification products that contain the genetic variations to be identified. Any suitable technique or method may be used for amplification. In general, the technique allows the (simultaneous) amplification of all the DNA sequences containing the genetic variations to be identified. In other words, where multiple genetic variations are to be analyzed, it is preferable to simultaneously amplify all of the corresponding target DNA regions (comprising the variations). Carrying out the amplification in a single step (or as few steps as possible) simplifies the method.

Analyzing a polynucleotide sample can be conducted in a number of ways. Preferably, the allele can optionally be subjected to an amplification step prior to performance of the detection step. Preferred amplification methods are selected from the group consisting of: the polymerase chain reaction (PCR), the ligase chain reaction (LCR), strand displacement amplification (SDA), cloning, and variations of the above (e.g. RT-PCR and allele specific amplification). A test nucleic acid sample can be amplified with primers that amplify a region known to comprise the target polymorphism(s), for example, from within the metabolic gene loci, either flanking the marker of interest (as required for PCR amplification) or directly overlapping the marker (as in allele specific oligonucleotide (ASO) hybridization). In a particularly preferred embodiment, the sample is hybridized with a set of primers, which hybridize 5′ and 3′ in a sense or antisense sequence to the vascular disease associated allele, and is subjected to a PCR amplification. Genomic DNA or mRNA can be used directly or indirectly, for example, to convert into cDNA. Alternatively, the region of interest can be cloned into a suitable vector and grown in sufficient quantity for analysis.

The nucleic acid may be amplified by conventional techniques, such as a polymerase chain reaction (PCR), to provide sufficient amounts for analysis. The use of the polymerase chain reaction is described in a variety of publications, including, e.g., “PCR Protocols (Methods in Molecular Biology)” (2000) J. M. S. Bartlett and D. Stirling, eds, Humana Press; and “PCR Applications: Protocols for Functional Genomics” (1999) Innis, Gelfand, and Sninsky, eds., Academic Press. Other methods for amplification of nucleic acids is ligase chain reaction (“LCR”), disclosed in European Application No. 320 308, isothermal amplification method, such as described in Walker et al., (Proc. Nat'l Acad. Sci. USA 89:392-396, 1992) or Strand Displacement Amplification or Repair Chain Reaction (RCR), transcription-based amplification systems (TAS), including nucleic acid sequence based amplification (NASBA) and 3SR. Kwoh et al., Proc. Nat'l Acad. Sci. USA 86:1173 (1989); Gingeras et al., PCT Application WO 88/10315, cyclic and non-cyclic synthesis of single-stranded RNA (“ssRNA”), ssDNA, and double-stranded DNA (dsDNA) (Davey et al., European Application No. 329 822 and Miller et al., PCT Application WO 89/06700, respectively) and di-nucleotide amplification (Wu et. al., Genomics 4:560 1989). Miller et al., PCT Application WO 89/06700 Alternative amplification methods include: self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197, PCT Application No. PCT/US87/00880), or any other nucleic acid amplification method (e.g., GB Application No. 2 202 328, and in PCT Application No. PCT/US89/01025), followed by the detection of the amplified molecules using techniques known to those of skill in the art. These detection schemes are useful for the detection of nucleic acid molecules if such molecules are present in very low numbers.

Once the region of interest has been amplified, the genetic variant of interest can be detected in the PCR product by nucleotide sequencing, by SSCP analysis, or any other method known in the art. In one embodiment, any of a variety of sequencing reactions known in the art can be used to directly sequence at least a portion of the gene of interest and detect allelic variants, e.g., mutations, by comparing the sequence of the sample sequence with the corresponding wild-type (control) sequence. Exemplary sequencing reactions include those based on techniques developed by Maxam and Gilbert (1997) Proc. Natl. Acad Sci, USA 74:560 or Sanger et al. (1977) Proc. Nat. Acad. Sci, 74:5463. It is also contemplated that any of a variety of automated sequencing procedures can be utilized when performing the subject assays (Biotechniques (1995) 19:448), including sequencing by mass spectrometry (see, for example, U.S. Pat. No. 5,547,835 and International Patent Application Publication Number WO94/16101, entitled DNA Sequencing by Mass Spectrometry by H. Koster; U.S. Pat. No. 5,547,835 and international patent application Publication No. WO 94/21822 entitled “DNA Sequencing by Mass Spectrometry Via Exonuclease Degradation” by H. Koster; U.S. Pat. No. 5,605,798 and International Patent Application No. PCT/US96/03651 entitled DNA Diagnostics Based on Mass Spectrometry by H. Koster; Cohen et al. (1996) Adv. Chromat. 36:127-162; and Griffin et al. (1993) Appl Biochem Bio. 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

In some embodiments of the present invention, variant sequences are detected using a PCR-based assay. In some embodiments, the PCR assay comprises the use of oligonucleotide primers that hybridize only to the variant or wild type allele (e.g., to the region of polymorphism or mutation). Both sets of primers are used to amplify a sample of DNA. If only the mutant primers result in a PCR product, then the patient has the mutant allele. If only the wild-type primers result in a PCR product, then the patient has the wild type allele.

In preferred embodiments of the present invention, variant sequences are detected using a hybridization assay. In a hybridization assay, the presence of absence of a given SNP or mutation is determined based on the ability of the DNA from the sample to hybridize to a complementary DNA molecule (e.g., a oligonucleotide probe). Parameters such as hybridization conditions, polymorphic primer length, and position of the polymorphism within the polymorphic primer may be chosen such that hybridization will not occur unless a polymorphism present in the primer(s) is also present in the sample nucleic acid. Those of ordinary skill in the art are well aware of how to select and vary such parameters. See, e.g., Saiki et al. (1986) Nature 324:163; and Saiki et al (1989) Proc. Natl. Acad. Sci. USA 86:6230.

Yet other sequencing methods are disclosed, e.g., in U.S. Pat. No. 5,580,732 entitled “Method of DNA Sequencing Employing A Mixed DNA-Polymer Chain Probe” and U.S. Pat. No. 5,571,676 entitled “Method For Mismatch-Directed In Vitro DNA Sequencing.”

In some cases, the presence of the specific allele in DNA from a subject can be shown by restriction enzyme analysis. For example, the specific nucleotide polymorphism can result in a nucleotide sequence comprising a restriction site that is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (see, e.g., Myers et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene of interest with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with 51 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249, Cotton et al. (1988) Proc. Natl. Acad. Sci. USA 85:4397; Saleeba et al. (1992) Methods Enzy. 217:286-295. In another embodiment, the control or sample nucleic acid is labeled for detection.

Over or under expression of a gene, in some cases, is correlated with a genomic polymorphism. The polymorphism can be present in an open reading frame (coded) region of the gene, in a “silent” region of the gene, in the promoter region, or in the 3′untranslated region of the transcript. Methods for determining polymorphisms are well known in the art and include, but are not limited to, the methods discussed below.

Detection of point mutations or additional base pair repeats (as required for the polymorphism) can be accomplished by molecular cloning of the specified allele and subsequent sequencing of that allele using techniques known in the art. Alternatively, the gene sequences can be amplified directly from a genomic DNA preparation from the sample using PCR, and the sequence composition is determined from the amplified product. As described more fully below, numerous methods are available for analyzing a subject's DNA for mutations at a given genetic locus such as the gene of interest.

A detection method is allele specific hybridization using probes overlapping the polymorphic site and having about 5, or alternatively 10, or alternatively 20, or alternatively 25, or alternatively 30 nucleotides around the polymorphic region. In another embodiment of the invention, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244.

Alternatively, various methods are known in the art that utilize oligonucleotide ligation as a means of detecting polymorphisms. See, e.g., Riley et al. (1990) Nucleic Acids Res. 18:2887-2890; and Delahunty et al. (1996) Am. J. Hum. Genet. 58:1239-1246.

In other embodiments, alterations in electrophoretic mobility are used to identify the particular allelic variant. For example, single strand conformation polymorphism (SSCP) may be used to detect differences in electrophoretic mobility between mutant and wild type nucleic acids (Orita et al. (1989) Proc Natl. Acad. Sci. USA 86:2766; Cotton (1993) Mutat. Res. 285:125-144 and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

In performing SSCP analysis, the PCR product may be digested with a restriction endonuclease that recognizes a sequence within the PCR product generated by using as a template a reference sequence, but does not recognize a corresponding PCR product generated by using as a template a variant sequence by virtue of the fact that the variant sequence no longer contains a recognition site for the restriction endonuclease.

In yet another embodiment, the identity of the allelic variant is obtained by analyzing the movement of a nucleic acid comprising the polymorphic region in polyacrylamide gels containing a gradient of denaturant, which is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al. (1989) Proc. Natl. Acad. Sci. USA 86:6230 and Wallace et al. (1979) Nucl. Acids Res. 6:3543). Such allele specific oligonucleotide hybridization techniques may be used for the detection of the nucleotide changes in the polylmorphic region of the gene of interest. For example, oligonucleotides having the nucleotide sequence of the specific allelic variant are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell. Probes 6:1).

In another embodiment, identification of the allelic variant is carried out using an oligonucleotide ligation assay (OLA), as described, e.g., in U.S. Pat. No. 4,998,617 and in Landegren, U. et al. Science 241:1077-1080 (1988). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson et al. (1990) Proc. Natl. Acad. Sci. (U.S.A.) 87:8923-8927). In this method, PCR is used to achieve the exponential amplification of target DNA, which is then detected using OLA.

Several techniques based on this OLA method have been developed and can be used to detect the specific allelic variant of the polymorphic region of the gene of interest. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. (1996) Nucleic Acids Res. 24: 3728, OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

In one embodiment, the single base polymorphism can be detected by using a specialized exonuclease-resistant nucleotide, as disclosed, e.g., in Mundy (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of the polymorphic site. Cohen et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet et al. (PCT Appln. No. 92/15712). This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet et al. supra, is preferably a heterogeneous phase assay, in which the primer or the target molecule is immobilized to a solid phase.

Recently, several primer-guided nucleotide incorporation procedures for assaying polymorphic sites in DNA have been described (Komher et al. (1989) Nucl. Acids. Res. 17:7779-7784; Sokolov (1990) Nucl. Acids Res. 18:3671; Syvanen et al. (1990) Genomics 8:684-692; Kuppuswamy et al. (1991) Proc. Natl. Acad. Sci. (U.S.A.) 88:1143-1147; Prezant et al. (1992) Hum. Mutat. 1:159-164; Ugozzoli et al. (1992) GATA 9:107-112; Nyren et al. (1993) Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen et al. (1993) Amer. J. Hum. Genet. 52:46-59).

In one aspect the invention provided for a panel of genetic markers selected from, but not limited to the genetic polymorphisms above. The panel comprises probes or primers that can be used to amplify and/or for determining the molecular structure of the polymorphisms identified above. The probes or primers can be attached or supported by a solid phase support such as, but not limited to a gene chip or microarray. The probes or primers can be detectably labeled. This aspect of the invention is a means to identify the genotype of a patient sample for the genes of interest identified above. In one aspect, the methods of the invention provided for a means of using the panel to identify or screen patient samples for the presence of the genetic marker identified herein. In one aspect, the various types of panels provided by the invention include, but are not limited to, those described herein. In one aspect, the panel contains the above identified probes or primers as wells as other, probes or primers. In an alternative aspect, the panel includes one or more of the above noted probes or primers and others. In a further aspect, the panel consist only of the above-noted probes or primers.

In one embodiment of the invention, probes are labeled with two fluorescent dye molecules to form so-called “molecular beacons” (Tyagi and Kramer (1996) Nat. Biotechnol. 14:303-8). Such molecular beacons signal binding to a complementary nucleic acid sequence through relief of intramolecular fluorescence quenching between dyes bound to opposing ends on an oligonucleotide probe. The use of molecular beacons for genotyping has been described (Kostrikis (1998) Science 279:1228-9) as has the use of multiple beacons simultaneously (Marras (1999) Genet. Anal. 14:151-6). A quenching molecule is useful with a particular fluorophore if it has sufficient spectral overlap to substantially inhibit fluorescence of the fluorophore when the two are held proximal to one another, such as in a molecular beacon, or when attached to the ends of an oligonucleotide probe from about 1 to about 25 nucleotides.

Labeled probes also can be used in conjunction with amplification of a polymorphism. (Holland et al. (1991) Proc. Natl. Acad. Sci. 88:7276-7280). U.S. Pat. No. 5,210,015 by Gelfand et al. describe fluorescence-based approaches to provide real time measurements of amplification products during PCR. Such approaches have either employed intercalating dyes (such as ethidium bromide) to indicate the amount of double-stranded DNA present, or they have employed probes containing fluorescence-quencher pairs (also referred to as the “Taq-Man” approach) where the probe is cleaved during amplification to release a fluorescent molecule whose concentration is proportional to the amount of double-stranded DNA present. During amplification, the probe is digested by the nuclease activity of a polymerase when hybridized to the target sequence to cause the fluorescent molecule to be separated from the quencher molecule, thereby causing fluorescence from the reporter molecule to appear. The Taq-Man approach uses a probe containing a reporter molecule-quencher molecule pair that specifically anneals to a region of a target polynucleotide containing the polymorphism.

Probes can be affixed to surfaces for use as “gene chips” or “microarray.” Such gene chips or microarrays can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarray” and similar technologies are know in the art. Examples of such include, but are not limited to LabCard (ACLARA Bio Sciences Inc.); GeneChip (Affymetrix, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc., San Diego WO 99/67641 and WO 00/39587); GEM (Incyte Microarray Systems); a high-throughput microarraying system that can dispense from 12 to 64 spots onto multiple glass slides (Intelligent Bio-Instruments); Molecular Biology Workstation and NanoChip (Nanogen, Inc.); a microfluidic glass chip (Orchid biosciences, Inc.); surface tension array (ProtoGene, Palo Alto, Calif. U.S. Pat. Nos. 6,001,311; 5,985,551; and 5,474,796), BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu Rev. Biomed. Eng. 4:129-153. Examples of “Gene chips” or a “microarray” are also described in US Patent Publ. Nos.: 2007-0111322, 2007-0099198, 2007-0084997, 2007-0059769 and 2007-0059765 and U.S. Pat. Nos. 7,138,506, 7,070,740, and 6,989,267.

In one aspect, “gene chips” or “microarrays” containing probes or primers for genes of the invention alone or in combination are prepared. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers may be detectably labeled thereby identifying the polymorphism in the gene(s) of interest. Alternatively, a chemical or biological reaction may be used to identify the probes or primers which hybridized with the DNA or RNA of the gene(s) of interest. The genotypes of the patient is then determined with the aid of the aforementioned apparatus and methods.

An allele may also be detected indirectly, e.g. by analyzing the protein product encoded by the DNA. For example, where the marker in question results in the translation of a mutant protein, the protein can be detected by any of a variety of protein detection methods. Such methods include immunodetection and biochemical tests, such as size fractionation, where the protein has a change in apparent molecular weight either through truncation, elongation, altered folding or altered post-translational modifications. Methods for measuring gene expression are also well known in the art and include, but are not limited to, immunological assays, nuclease protection assays, northern blots, in situ hybridization, reverse transcriptase Polymerase Chain Reaction (RT-PCR), Real-Time Polymerase Chain Reaction, expressed sequence tag (EST) sequencing, cDNA microarray hybridization or gene chip analysis, statistical analysis of microarrays (SAM), subtractive cloning, Serial Analysis of Gene Expression (SAGE), Massively Parallel Signature Sequencing (MPSS), and Sequencing-By-Synthesis (SBS). See for example, Carulli et al., (1998) J. Cell. Biochem. 72 (S30-31): 286-296; Galante et al., (2007) Bioinformatics, Advance Access (Feb. 3, 2007).

SAGE, MPSS, and SBS are non-array based assays that determine the expression level of genes by measuring the frequency of sequence tags derived from polyadenylated transcripts. SAGE allows for the analysis of overall gene expression patterns with digital analysis. SAGE does not require a preexisting clone and can used to identify and quantitate new genes as well as known genes. Velculescu et al., (1995) Science 270(5235):484-487; Velculescu (1997) Cell 88(2):243-251.

MPSS technology allows for analyses of the expression level of virtually all genes in a sample by counting the number of individual mRNA molecules produced from each gene. As with SAGE, MPSS does not require that genes be identified and characterized prior to conducting an experiment. MPSS has a sensitivity that allows for detection of a few molecules of mRNA per cell. Brenner et al. (2000) Nat. Biotechnol. 18:630-634; Reinartz et al., (2002) Brief Funct. Genomic Proteomic 1: 95-104.

SBS allows analysis of gene expression by determining the differential expression of gene products present in sample by detection of nucleotide incorporation during a primer-directed polymerase extension reaction.

SAGE, MPSS, and SBS allow for generation of datasets in a digital format that simplifies management and analysis of the data. The data generated from these analyses can be analyzed using publicly available databases such as Sage Genie (Boon et al., (2002) PNAS 99:11287-92), SAGEmap (Lash et al., (2000) Genome Res 10:1051-1060), and Automatic Correspondence of Tags and Genes (ACTG) (Galante (2007), supra). The data can also be analyzed using databases constructed using in house computers (Blackshaw et al. (2004) PLoS Biol, 2:E247; Silva et al. (2004) Nucleic Acids Res 32:6104-6110)).

Moreover, it will be understood that any of the above methods for detecting alterations in a gene or gene product or polymorphic variants can be used to monitor the course of treatment or therapy.

The methods described herein may be performed, for example, by utilizing pre-packaged diagnostic kits, such as those described below, comprising at least one probe or primer nucleic acid described herein, which may be conveniently used, e.g., to determine whether a subject has or may have a greater or lower response to SSRI treatments.

Diagnostic procedures can also be performed in situ directly upon samples from, such that no nucleic acid purification is necessary. Nucleic acid reagents can be used as probes and/or primers for such in situ procedures (see, for example, Nuovo (1992) “PCR 1N SITU HYBRIDIZATION: PROTOCOLS AND APPLICATIONS”, Raven Press, NY).

In addition to methods that focus primarily on the detection of one nucleic acid sequence, profiles can also be assessed in such detection schemes. Fingerprint profiles can be generated, for example, by utilizing a differential display procedure, Northern analysis and/or RT-PCR.

### Nucleic Acids

In one aspect, the nucleic acid sequences of the gene's allelic variants, or portions thereof, can be the basis for probes or primers, e.g., in methods and compositions for determining and identifying the allele present at the gene of interest's locus, more particularly to identity the allelic variant of a polymorphic region(s). Thus, they can be used in the methods of the invention to determine which therapy is most likely to affect or not affect an individual's disease or disorder, such as to diagnose and prognose disease progression as well as select the most effective treatment among treatment options. Probes can be used to directly determine the genotype of the sample or can be used simultaneously with or subsequent to amplification.

The methods of the invention can use nucleic acids isolated from vertebrates. In one aspect, the vertebrate nucleic acids are mammalian nucleic acids. In a further aspect, the nucleic acids used in the methods of the invention are human nucleic acids.

Primers and probes for use in the methods of the invention are nucleic acids that hybridize to a nucleic acid sequence which is adjacent to the region of interest or which covers the region of interest and is extended. A primer or probe can be used alone in a detection method, or a can be used together with at least one other primer or probe in a detection method. Primers can also be used to amplify at least a portion of a nucleic acid. Probes for use in the methods of the invention are nucleic acids which hybridize to the region of interest and which are generally are not further extended. Probes may be further labeled, for example by nick translation, Klenow fill-in reaction, PCR or other methods known in the art, including those described herein). For example, a probe is a nucleic acid which hybridizes to the polymorphic region of the gene of interest, and which by hybridization or absence of hybridization to the DNA of a subject will be indicative of the identity of the allelic variant of the polymorphic region of the gene of interest. Probes and primers of the present invention, their preparation and/or labeling are described in Green and Sambrook (2012). Primers and Probes useful in the methods described herein are found in Table 1.

In one embodiment, primers and probes comprise a nucleotide sequence which comprises a region having a nucleotide sequence which hybridizes under stringent conditions to about 5 through about 100 consecutive nucleotides, more particularly about: 6, 8, 10, 12, 15, 20, 25, 30, 35, 40, 45, 50, 60, or 75 consecutive nucleotides of the gene of interest. Length of the primer or probe used will depend, in part, on the nature of the assay used and the hybridization conditions employed.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to nucleotide sequences located about 150 to about 350 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified.

Yet other preferred primers of the invention are nucleic acids that are capable of selectively hybridizing to an allelic variant of a polymorphic region of the gene of interest. Thus, such primers can be specific for the gene of interest sequence, so long as they have a nucleotide sequence that is capable of hybridizing to the gene of interest.

The probe or primer may further comprises a label attached thereto, which, e.g., is capable of being detected, e.g. the label group is selected from amongst radioisotopes, fluorescent compounds, enzymes, and enzyme co-factors.

Additionally, the isolated nucleic acids used as probes or primers may be modified to become more stable. Exemplary nucleic acid molecules that are modified include phosphoramidate, phosphothioate and methylphosphonate analogs of DNA (see also U.S. Pat. Nos. 5,176,996; 5,264,564 and 5,256,775).

The nucleic acids used in the methods of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane. See, e.g., Letsinger et al., (1989) Proc. Natl. Acad. Sci. U.S.A. 86:6553-6556; Lemaitre et al., (1987) Proc. Natl. Acad. Sci. 84:648-652; and PCT Publication No. WO 88/09810, published Dec. 15, 1988), hybridization-triggered cleavage agents, (see, e.g., Krol et al., (1988) BioTechniques 6:958-976) or intercalating agents (see, e.g., Zon (1988) Pharm. Res. 5:539-549. To this end, the nucleic acid used in the methods of the invention may be conjugated to another molecule, e.g., a peptide, hybridization triggered cross-linking agent, transport agent, hybridization-triggered cleavage agent, etc.

The isolated nucleic acids used in the methods of the invention can also comprise at least one modified sugar moiety selected from the group including but not limited to arabinose, 2-fluoroarabinose, xylulose, and hexose or, alternatively, comprise at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The nucleic acids, or fragments thereof, to be used in the methods of the invention can be prepared according to methods known in the art and described, e.g., in Sambrook and Russel (2001) supra. For example, discrete fragments of the DNA can be prepared and cloned using restriction enzymes. Alternatively, discrete fragments can be prepared using the Polymerase Chain Reaction (PCR) using primers having an appropriate sequence under the manufacturer's conditions, (described above).

Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (1988) Nucl. Acids Res. 16:3209, methylphosphonate oligonucleotides can be prepared by use of controlled pore glass polymer supports. Sarin et al. (1988) Proc. Natl. Acad. Sci. U.S.A. 85:7448-7451.

TABLE 1

| | | Forward | | Reverse |
|---|---|---|---|---|
| Marker | Forward | Primer | Reverse | Primer |
| Name | Primer | Sequence | Primer | Sequence |
| 5-HTTLPR | rs25531. | SEQ. ID | SEQ. ID NO: | SEQ. ID |
| | fwd.1 | NO: 29 | 5-HTTLR. | NO: 3 |
| | | CTCCCTGTAC | rev.1 | TGCAGGGGG |
| | | CCCTCCTAGG | | ATGCTGGAA |
| | rs25531. | SEQ. ID | SEQ. ID NO: | SEQ. ID |
| | fwd.1 | NO: 30 | 5-HTTLR. | NO: 31 |
| | | CTCCCTGTAC | rev.2 | AGAGGTGCA |
| | | CCCTCCTAGG | | GGGGGATG |
| | rs25531. | SEQ. ID | SEQ. ID NO: | SEQ. ID |
| | fwd.3 | NO: 2 | 5-HTTLR. | NO: 3 |
| | | CAACTCCCTG | rev.1 | TGCAGGGGG |
| | | TACCCCTCCT | | ATGCTGGAA |
| rs25531 | rs25531. | SEQ. ID | SEQ. ID NO: | SEQ. ID |
| | fwd.1 | NO: 35 | rs25531. | NO: 32 |
| | | CTCCCTGTAC | rev.1 | GGTGCAGGG |
| | | CCCTCCTAGG | | GAGATGCTG |
| | rs25531. | SEQ. ID | SEQ. ID NO: | SEQ. ID |
| | fwd.2 | NO: 45 | rs25531. | NO: 33 |
| | | CTCCCAGCAA | rev.1 | GGTGCAGGG |
| | | CTCCCTGTAC | | GAGATGCTG |
| | rs25531. | SEQ. ID | SEQ. ID NO: | SEQ. ID |
| | fwd.3 | NO: 6 | rs25531. | NO: 34 |
| | | CAACTCCCTG | rev.1 | GGTGCAGGG |
| | | TACCCCTCCT | | GAGATGCTG |
| | rs25531. | SEQ. ID | SEQ. ID NO: | SEQ. ID |
| | fwd.1 | NO: 36 | rs25531. | NO: 7 |
| | | CTCCCTGTAC | rev.2 | GAGATGCTG |
| | | CCCTCCTAGG | | GGGGGGCT |
| | rs25531. | SEQ. ID | SEQ. ID NO: | SEQ. ID |
| | fwd.2 | NO: 37 | rs25531. | NO: 7 |
| | | CTCCCAGCAA | rev.2 | GAGATGCTG |
| | | CTCCCTGTAC | | GGGGGGCT |
| | rs25531. | SEQ. ID | SEQ. ID NO: | SEQ. ID |
| | fwd.3 | NO: 6 | rs25531. | NO: 7 |
| | | CAACTCCCTG | rev.2 | GAGATGCTG |
| | | TACCCCTCCT | | GGGGGGCT |
| | rs25531. | SEQ. ID | SEQ. ID NO: | SEQ. ID |
| | fwd.3 | NO: 6 | rs25531. | NO: 38 |
| | | CAACTCCCTG | rev.4 | CTGGGGGTG |
| | | TACCCCTCCT | | CAGGGGA |

TABLE 2

| Marker | VIC Probe | VIC Probe | FAM Probe | FAM Probe |
|---|---|---|---|---|
| Name | Name | sequence | Name | Sequence |
| 5-HTTLPR | 5_HTTLR.a_V | SEQ. ID | 5_HTTLR. | SEQ. ID |
| | | NO: 39 | a_F | NO: 42 |
| | | CCAGCATCT | | CTTCACCCC |
| | | CCCCT | | TCGCGG |
| | 5_HTTLR.b_V | SEQ. ID | 5_HTTLR. | SEQ. ID |
| | | NO: 5 | b_F | NO: 4 |
| | | AGCCCCCCC | | TCCTGCATC |
| | | AGCATCTC | | CCCCATTAT |
| | | | | CC |
| | 5_HTTLPR. | SEQ. ID | 5_HTTLPR. | SEQ. ID |
| | c_V | NO: 40 | c_F | NO: 43 |
| | | TGCAGCCCC | | TCCCCCCCT |
| | | CCCAGCATC | | TCACCCCTC |
| | | TCCC | | GCGGCATCC |
| | | | | C |
| rs25531 | rs25531_V | SEQ. ID | rs25531_F | SEQ. ID |
| | | NO: 41 | | NO: 44 |
| | | TGCACCCCC | | CTGCACCCC |
| | | GGCA | | CAGC |
| | rs25531.b_V | SEQ. ID | rs25531. | SEQ. ID |
| | | NO: 9 | b_F | NO: 8 |
| | | CTGCACCCC | | CCTGCACCC |
| | | CGGCA | | CCAGC |

### Kits

As set forth herein, the invention provides diagnostic methods for determining the type of allelic variant of a polymorphic region present in the gene of interest or the expression level of a gene of interest. In some embodiments, the methods use probes or primers comprising nucleotide sequences which are complementary to the polymorphic region of the gene of interest. Accordingly, the invention provides kits for performing these methods as well as instructions for carrying out the methods of this invention such as collecting tissue and/or performing the screen, and/or analyzing the results, and/or administration of an effective amount of the therapies described above.

In an embodiment, the invention provides a kit for determining whether a subject responds to SSRI treatment or alternatively one of various treatment options. The kits contain one of more of the compositions described above and instructions for use. As an example only, the invention also provides kits for determining response to SSRI treatment containing a first and a second oligonucleotide specific for the polymorphic region of the gene. Oligonucleotides “specific for” a genetic locus bind either to the polymorphic region of the locus or bind adjacent to the polymorphic region of the locus. For oligonucleotides that are to be used as primers for amplification, primers are adjacent if they are sufficiently close to be used to produce a polynucleotide comprising the polymorphic region. In one embodiment, oligonucleotides are adjacent if they bind within about 1-2 kb, and preferably less than 1 kb from the polymorphism. Specific oligonucleotides are capable of hybridizing to a sequence, and under suitable conditions will not bind to a sequence efficiently differing by a single nucleotide.

The kit can comprise at least one probe or primer which is capable of specifically hybridizing to the polymorphic region of the gene of interest and instructions for use. The kits preferably comprise at least one of the above described nucleic acids. Preferred kits for amplifying at least a portion of the gene of interest comprise two primers and two probes, at least one of probe is capable of binding to the allelic variant sequence. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the preferred surface is silica or glass. In another embodiment, the surface is a metal electrode.

Yet other kits of the invention comprise at least one reagent necessary to perform the assay. For example, the kit can comprise an enzyme. Alternatively the kit can comprise a buffer or any other necessary reagent.

Conditions for incubating a nucleic acid probe with a test sample depend on the format employed in the assay, the detection methods used, and the type and nature of the nucleic acid probe used in the assay. One skilled in the art will recognize that any one of the commonly available hybridization, amplification or immunological assay formats can readily be adapted to employ the nucleic acid probes for use in the present invention. Examples of such assays can be found in Chard (1986) AN INTRODUCTION TO RADIOIMMUNOASSAY AND RELATED TECHNIQUES Elsevier Science Publishers, Amsterdam, The Netherlands; Bullock et al. TECHNIQUES IN IMMUNOCYTOCHEMISTRY Academic Press, Orlando, Fla. Vol. 1 (1982), Vol. 2 (1983), Vol. 3 (1985); Tijssen, PRACTICE AND THEORY OF IMMUNOASSAYS: LABORATORY TECHNIQUES IN BIOCHEMISTRY AND MOLECULAR BIOLOGY, Elsevier Science Publishers, Amsterdam, The Netherlands (1985).

The test samples used in the diagnostic kits include cells, protein or membrane extracts of cells, or biological fluids such as sputum, blood, serum, plasma, or urine. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region or the expression levels of the gene of interest.

As amenable, these suggested kit components may be packaged in a manner customary for use by those of skill in the art. For example, these suggested kit components may be provided in solution or as a liquid dispersion or the like.

Other Uses for the Nucleic Acids of the Invention

The identification of the allele of the gene of interest can also be useful for identifying an individual among other individuals from the same species. For example, DNA sequences can be used as a fingerprint for detection of different individuals within the same species. Thompson and Thompson, Eds., (1991) GENETICS IN MEDICINE, W B Saunders Co., Philadelphia, Pa. This is useful, e.g., in forensic studies.

The invention now being generally described, it will be more readily understood by reference to the following examples which are included merely for purposes of illustration of certain aspects and embodiments of the present invention, and are not intended to limit the invention.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, microbiology, virology, recombinant DNA technology, peptide synthesis in solution, solid phase peptide synthesis, histology and immunology. Such procedures are described, for example, in the following texts that are incorporated by reference:
(i) Green M R, Sambrook J, Molecular Cloning: A Laboratory Manual, Cold Spring Harbor Laboratories Press, New York, Fourth Edition (2012), whole of Vols I, II, and III;
(ii) DNA Cloning: A Practical Approach, Vols. I-IV (D. M. Glover, ed., 1995), Oxford University Press, whole of text;
(iii) Oligonucleotide Synthesis: Methods and Application (P Herdewijn, ed., 2010) Humana Press, Oxford, whole of text;
(iv) Nucleic Acid Hybridization: A Practical Approach (B. D. Hames & S. J. Higgins, eds., 1985) IRL Press, Oxford, whole of text;
(v) van Pelt-Verkuil, E, van Belkum, A, Hays, J P. Principles and Technical Aspects of PCR Amplification (2010) Springer, whole of text;
(vi) Perbal, B., A Practical Guide to Molecular Cloning, 3rd Ed. (2008);
(vii) Gene Synthesis: Methods and Protocols (J Peccoud, ed. 2012) Humana Press, whole of text;
(viii) PCR Primer Design (Methods in Molecular Biology). (A Yuryev. ed., 2010), Humana Press, Oxford, whole of text.

### EXAMPLES

### Example 1

### SNP Genotyping

### Template DNA

Genomic DNA from Coriell Cell lines and genomic DNA extracted from Saliva samples using Pathway standard protocol (L-0024.006) were normalized to 50 ng/μL in 96 well plates.

### Primers, Probes and Assay Assembly

All the primers were synthesized by IDT using standard desalted oligo protocol. TaqMan MGB probes were designed and manufactured by life Technologies. Two probes of each assay were labeled either FAM or VIC 5′ reporter dye (Applied Biosystems). Each TaqMan assay was prepared as 20× stock containing 100 nM primers (both forward and reverse), 100 μM of TaqMan probes (both FAM and VIC) in 1×TE buffer (PH 8.0) to give a final concentration of 18 nM per primer and 4 μM per probe (or desirable concentration).

### Pre-Amplification of Template DNA

Each 5 μl multiplex pre-amplification reaction consisted of 2× QIAGEN Multiplex PCR Master Mix, 0.2× pooled sequence-specific gene assay mix and 1.25 μL of template DNA (50 ng/μL). Pre-amplification no template control (NTC) was set up in parallel with template DNA, but without target DNA. Pre-amplification was performed using BioRad T100 system with thermo cycling conditions of 95° C. for 15 minutes, followed by 14 cycles of 95° C. for 15 seconds and 60° C. for 4 minutes. All pre-amplification reactions were diluted 1:5 with 1×TE buffer (PH 8.0) to give a total volume of 25 μL.

### Example 2

### TaqMan SNP Genotyping

SNP Genotyping reactions were set up by preparing the assays and samples separately according to manufacturer's instruction. For each assay, 5.25 μL of a 10× assay reaction was prepared by mixing 2.5 μL 2× assay loading reagent(Fluidigm), 2.5 μL 20× assembled TaqMan assay and 0.25 μL 50×ROX (Life Technogies). For each sample, 6 μL of sample solution was prepared by mixing 2.5 μL diluted pre-amplification reaction and 3.5 μL pre-sample mix containing 2× TagMan genotyping Mastermix (life Technologies), 0.3 μL 20× Genotyping loading buffer(Fluidigm), 0.06 μL AmpliTaq Gold DNA polymerase (5U/μL, Life technologies) and 0.14 μL nucleotide free H2O. For NTC, nucleotide free H2O was used instead of DNA template. Four μL of a 10× TaqMan assay mix and 5 μL of GT-sample solution were loaded into assigned assay or sample inlets of the primed Fluidigm 96.96 Dynamic Array™, respectively. The Fluidigm 96.96 Dynamic Array™ was placed on the IFC controller for loading and mixing. The chip was then placed on the BioMark™ instrument for thermal cycling: 2 minutes at 50° C., 30 minutes at 70° C., 10 minutes at 25° C., 2 minutes at 50° C., and 10 min at 95° C. followed by 40 cycles of a 2-step amplification profile of 15 seconds at 95° C. and 1 min at 60° C. The data was analyzed using the Fluidigm genotyping analysis software v3.0.2 which produces a genotype call for each sample/assay combination.

### Example 3

### 5-HTTLPR PCR Optimization

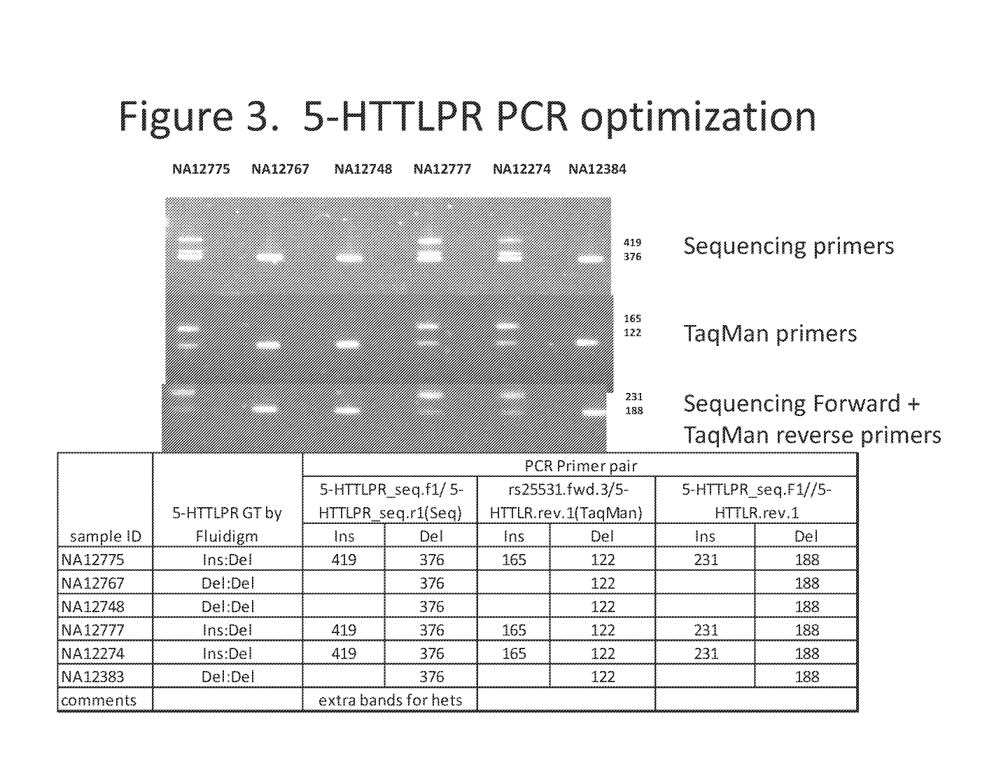

Three paired primers were used to PCR the unique region surrounding rs25531 (5-HTTLPR allele region). Six Coriell cell line samples (NA12775, NA12767, NA12748, NA12777, NA12274 and NA12383) were used for testing the specificity of the primers. Each 10 μL PCR amplification reaction consisted of 5 μL 2× GC buffer I, 0.1 μL TaKaRa LA Taq, 1.6 μL 2.5 μM dNTP mixtures, 0.2 μL 10 μM primers (both forward and reverse), 0.9 μL nucleotide free H2O and 2 μL of template DNA. The PCR amplification was performed using BioRad T100 System with thermal cycling conditions of 94° C. for 1 minute, followed by 30 cycles of 94° C. for 30 seconds, 60° C. for 30 seconds, and 72° C. for 1 minute and hold at 72° C. for 7 minutes. 2 μL PCR products were then loaded into 1% agrose gel and run for 20 minutes. The FIG. 3 showed the performance of the three primer pairs. TaqMan primer pairs (rs25531.fwd.3 and 5-HTTLPT_rev.1) used in 5-HTTLPR Fludigm assay as well as the sequencing primer pair (5-HTTLPR_seq.F1 and 5-HTTLPR_rev.1) produced specific PCR product within the L/S allele breakpoint. The sequencing primer pairs (5-HTTLPR_seq.f1/5-HTTLPR_seq.r1) produced an unspecific product in the L allele region. Therefore, 5-HTTLPR Taqman assay containing TaqMan primer pair could accurately detected the 16 repeat long allele (165 bp) and 14 repeat short allele (122 bp) in the 5-HTTLPR promoter region. The sequencing primer pair ((5-HTTLPR_seq.F1 and 5-HTTLPR_rev.1) amplified a 231-bp product for the long allele and 188-bp product for the short S allele. Sequencing confirmed the 5-HTTLPR allele genotypes.

### Example 4

### Detection of the 5-HTTLPR Polymorphism (rs25531) Using Novel Probe

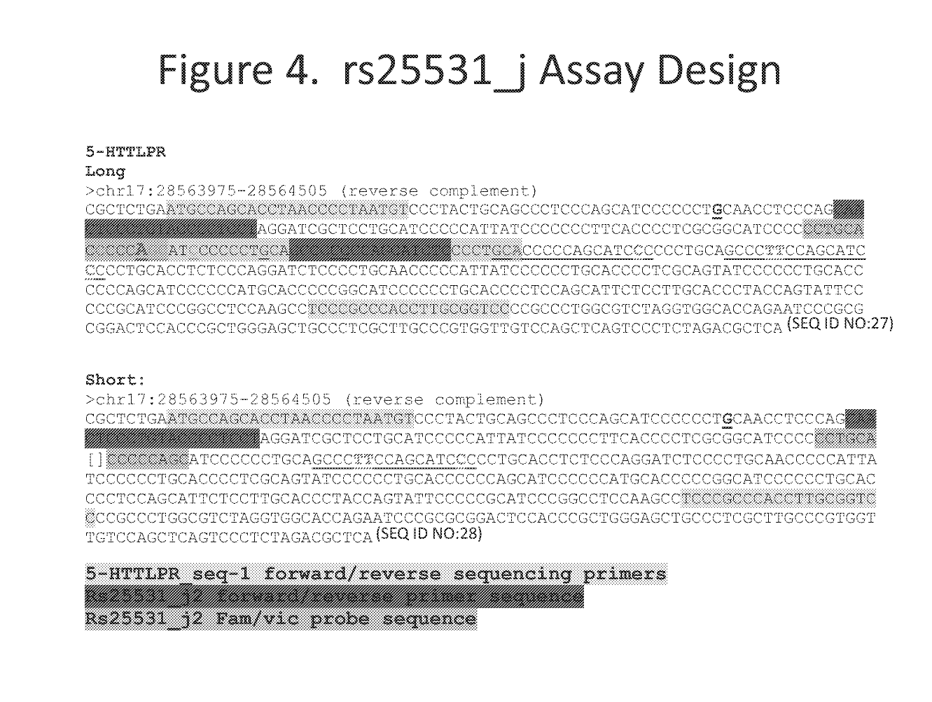

The SNP rs25531 is only present in the long or insertion (INS) allele of 5-HTTLPR but not the short or deletion (DEL) allele. For rs25531 TaqMan assay, the reverse assay and sequencing primers were designed to be specific to the long form (INS allele) of 5-HTTLPR, so only 5-HTTLPR INS alleles were amplified during PCR amplifications. For samples with 5-HTTLPR S-allele genotypes (Del:Del), the rs25531 should generate amplicons (no calls), for samples with 5-HTTLPR heterozygous genotypes (Ins:Del), the rs25531 assay only generated homozygous (LL) genotypes FIG. 4).

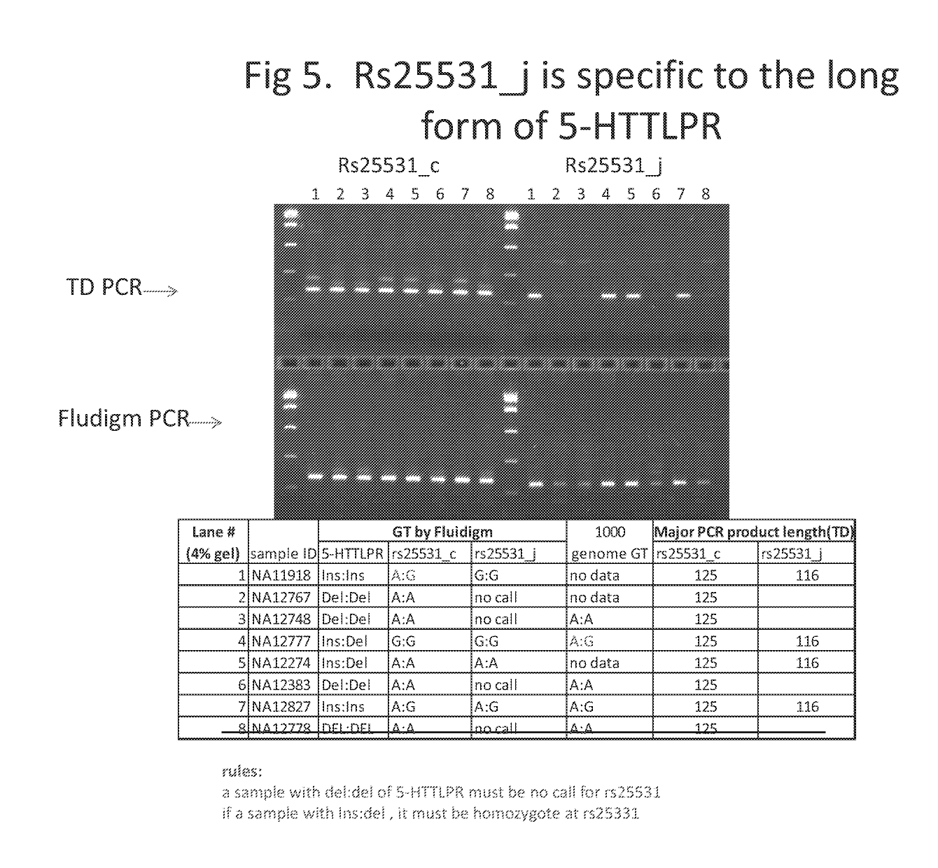

Eight Coriell cell line samples (NA11918, NA12767, NA12748, NA12777, NA12274, NA12383, NA12827, and NA12778) carried three possible 5-HTTLPR genotypes were chosen to test the specificity of the two reverse primers(rs25531.rev.1 and rs25531.rev.2). Two different PCR conditions: TD(15 min at 95° C., followed by 10cycles of 3-step amplification profile of 30 seconds at 94 C, 45 seconds at 65° C.-1° C. per cycle and 1 minute at 72° C., followed by 35 cycles of 3-step amplification profile of 30 seconds at 94° C., 45 seconds at 55° C. and 1 minute at 72° C., and then hold 7 minutes at 72° C.) and Fluidigm PCR (2 minutes at 50° C., 30 minutes at 70° C., 10 minutes at 25° C., 2 minutes at 50° C., and 10 min at 95° C., followed by 40 cycles of a 2-step amplification profile of 15 seconds at 95° C. and 1 min at 60° C.) were used for this study. The FIG. 5 showed that rs25531.rev.2 (rs25531_j) was specific to the long allele while rs25531.rev.1 (rs25531_c) was not. Weak or noise signals (due to high similarity in the region) for the short allele in the Fludigm PCRs could be separated from the major clusters in the rs25531_J assay (FIG. 5). The sequence data for rs25531 were 100% identical to the genotyping run data. (FIG. 5 and TABLE 3).

TABLE 3

| | | | sequencing results | | |
|---|---|---|---|---|---|
| | | | | | rs25531 |
| | Expected genotypes | | | rs25531 | position |
| Sample ID | 5-HTTLPR | rs25531 | 5-HTTLPR | INS allele | DEL allele |
| NA12058 | Ins:Del | G:G | Ins:Del | G:G | A:A |
| NA12274 | Ins:Del | A:A | Ins:Del | A:A | A:A |
| NA12775 | Ins:Del | A:A | Ins:Del | A:A | A:A |
| NA12777 | Ins:Del | G:G | Ins:Del | G:G | A:A |

1. A reaction mixture for amplicon production comprising:
an oligonucleotide comprising a nucleotide sequence capable of specifically hybridizing under stringent conditions to at least 10 consecutive nucleotides of a promoter region of a human SLC6A4 gene having the nucleotide sequence of SEQ ID NO: 10, or a complement thereof;
a dNTP mixture consisting of dTTP, dGTP, dATP, and dCTP, or analogs thereof.

2. The reaction mixture of claim 1, wherein the oligonucleotide is capable of specifically hybridizing to at least 10 consecutive nucleotides of the sequence of SEQ ID NO: 1, or a complement thereof.

3. The reaction mixture of claim 1, wherein the oligonucleotide comprises the sequence of any one of SEQ ID NOs: 2-9 and 29-44, or a complement thereof.

4.-62. (canceled)

63. The reaction mixture of claim 1, wherein the oligonucleotide comprises the sequence of SEQ ID NO: 5 or a complement thereof.

64. The reaction mixture of claim 63, further comprising a second oligonucleotide that comprises the sequence of SEQ ID NO: 4.

65. The reaction mixture of claim 1, further comprising one or more of the following reagents:
a) a DNA polymerase;
b) a buffer;
c) a surfactant;
e) a reducing agent; and
e) a chemical denaturant.

66. A method for selecting a mental health treatment for an individual comprising:
assaying for the allele status of the SLC6A4 locus, and
selecting an SSRI pharmaceutical if the allele status is homozygous for a long allele of the SLC6A4 locus (SLC6A4 L allele), or selecting a non-SSRI pharmaceutical if the allele status is not homozygous for an SLC6A4 L allele,
wherein said assaying comprises obtaining a biological sample from the individual, and contacting the obtained sample with an oligonucleotide comprising the sequence of any one of SEQ ID NOs: 2-9 and 29-44, or a complement thereof.

67. The method of claim 66, wherein the SSRI pharmaceutical is selected from the group consisting of fluoxetine, fluvoxamine, citalopram, cericlamine, dapoxetine, escitalopram, femoxetine, indalpine, paroxetine, sertraline, paroxetine, ifoxetine, cyanodothiepin, zimelidine, and litoxetine.

68. The method of claim 66, wherein said assaying for SLC6A4 allele status comprises obtaining a sample from the individual, contacting the obtained sample with a first probe that detects an SLC6A4 L allele comprising a sequence of SEQ ID NO: 1, and contacting the sample with a second probe that detects a sequence located outside of the sequence of SEQ ID NO: 1 of said SLC6A4 locus.

69. The method of claim 68, wherein the first probe comprises the sequence of SEQ ID NO: 5 or a complement thereof.

70. The method of claim 68, wherein the second probe comprises the sequence of SEQ ID NO: 4.

71. The method of claim 66, further comprising detecting a single nucleotide polymorphism (SNP) in the 5-hydroxytryptamine-transporter-gene-linked polymorphic region (5-HTTLPR region) of the SLC6A4 locus.

72. The method of claim 71, wherein the polymorphism is rs25531.

73. The method of claim 72, further comprising selecting a non-SSRI pharmaceutical if the allele status at rs25531 is the nucleobase guanine (“G”) even if the biological sample is homozygous for the L allele.

74. The method of claim 66, wherein said assaying for SLC6A4 allele status comprises obtaining a sample from said individual, contacting said sample with the reaction mixture of claim 1.

75. The method of claim 66, wherein the sample is derived
from blood, including serum, lymphocytes, lymphoblastoid cells, fibroblasts, platelets, mononuclear cells or other blood cells, or
from saliva, liver, kidney, pancreas or heart, urine, or
from any other tissue, fluid, cell or cell line derived from the human body.

76. A diagnostic kit comprising the reaction mixture for amplicon production of claim 1.

77. The diagnostic kit of claim 76, further comprising one or more of the followings:
a) instructions for performing the amplicon production;
b) one or more positive controls and/or negative controls.
